SEQUENCE LISTING (6 sequences)

SEQ ID NO: 1 - protein, 15 aa, Homo sapiens
ILPSDIMDFLVKNTP

SEQ ID NO: 2 - protein, 19 aa, Homo sapiens
VLLKLASPELERLIIQSSN

SEQ ID NO: 3 - protein, 20 aa, Homo sapiens
MEPVDPRLEPWKHPGSQPKT

SEQ ID NO: 4 - protein, 19 aa, Homo sapiens
PSFPTQRTSKTLKVLPPIT

SEQ ID NO: 5 - DNA, 34 bp, Artificial Sequence
  Synthetic
gactggatccctgaaatcagattcagacaataac

SEQ ID NO: 6 - DNA, 33 bp, Artificial Sequence
  Synthetic
caaggcggccgcaagacccaatccttcaccaag

(19) United States
(12) Patent Application Publication
(10) Pub. No.: US 2015/0025045 A1
(43) Pub. Date: Jan. 22, 2015

(54) CBP AND p300-MEDIATED TRANSCRIPTION MODULATORS AND RELATED METHODS

(71) Applicants: Anna Mapp, Ann Arbor, MI (US); Quintin Pan, Columbus, OH (US)

(72) Inventors: Anna Mapp, Ann Arbor, MI (US); Quintin Pan, Columbus, OH (US)

(73) Assignees: THE OHIO STATE UNIVERSITY, Columbus, OH (US); THE REGENTS OF THE UNIVERSITY OF MICHIGAN, Ann Arbor, MI (US)

(21) Appl. No.: 14/344,068

(22) PCT Filed: Sep. 14, 2012
(86) PCT No.: PCT/US2012/055547

§ 371 Date: Sep. 26, 2014

Related U.S. Application Data
(60) Provisional application No. 61/535,845, filed on Sep. 16, 2011.

(51) Int. Cl. C07D 261/02; A61K 45/06; C12N 15/63; A61K 31/655

(52) U.S. Cl. CPC C07D 261/02; A61K 31/655; A61K 45/06; C12N 15/63
USPC 514/151; 435/375

(57) ABSTRACT

The present invention relates to gene regulation. In particular, the present invention provides small compounds capable of modulating p300 and/or CBP-mediated transcription and related methods of therapeutic and research use. In addition, the present invention provides methods for treating conditions associated with aberrant p300 and/or CBP-mediated transcription with p300 and/or CBP-mediated transcription modulators (e.g., p300 and/or CBP-mediated transcription inhibitors).

### CROSS-REFERENCE TO RELATED APPLICATIONS

This application claims priority to pending U.S. Provisional Patent Application No. 61/535,845, filed Sep. 16, 2011.

### STATEMENT REGARDING FEDERALLY SPONSORED RESEARCH OR DEVELOPMENT

This invention was made with support provided by U.S. Department of Health & Human Services grant number GM06553. The government has certain rights in the invention.

### FIELD OF THE INVENTION

The present invention relates to gene regulation. In particular, the present invention provides small compounds capable of modulating p300 and/or CBP-mediated transcription and related methods of therapeutic and research use. In addition, the present invention provides methods for treating conditions associated with aberrant p300 and/or CBP-mediated transcription with p300 and/or CBP-mediated transcription modulators (e.g., p300 and/or CBP-mediated transcription inhibitors).

### BACKGROUND OF THE INVENTION

p300/CBP (CREB-binding protein) is a histone acetyltransferase that promotes transcription through acetylation of the histones, thereby undoing the nucleosome and exposing DNA regions for transcription. The p300/CBP protein consists of two subdomains known as the N and C subdomains, with a portion of the C domain containing a loop that caps both ends of the N domain. Another important feature of the structure is the presence of a tunnel and a pocket that interact with lysine side chains, which leads to acetylation of these lysines in the histone tail.

p300 activates transcription through direct interaction with RNA polymerase II and transcription factors. Since p300/CBP acetyltransferases play important roles in many biological processes, such as cell proliferation and differentiation, much research is being undertaken to see how they function in cancer. For example, research has shown that p300/CBP may contribute to p53 degradation and therefore help cells resist apoptosis, which would therefore lead to cancer (see, e.g., Goodman, 2000).

Because of the association of mutant p300/CBP with cancer, as well as its function in other diseases including cardiac disease and diabetes, inhibition of these proteins may represent an avenue for therapeutic treatments. Accordingly, there is a desire to find a method of preventing the development of the diseases related p300/CBP.

### SUMMARY OF THE INVENTION

Amphipathic isoxazolidines are able to functionally replace the transcriptional activation domains of endogenous transcriptional activators. In addition, in vitro binding studies suggested that a key binding partner of these molecules is the CREB Binding Protein (CBP), more specifically the KIX domain within this protein. Experiments conducted during the course of developing embodiments for the present invention demonstrated that CBP plays an essential role in the ability of isoxazolidine transcriptional activation domains to activate transcription in cells. Consistent with this model, isoxazolidines were shown to be able to function as competitive inhibitors of the activators MLL and Jun, both of which utilize a binding interaction with KIX to up-regulate transcription. Further, it was shown that modification of the N2 side chain produced analogs with enhanced potency against Jun-mediated transcription, although increased cytotoxicity was also observed.

In addition, Nanog-regulated genes are dependent upon p300. A unique characteristic of cancer-initiating cells (CICs or cancer stem cells) relative to embryonic or adult stem cells is a reliance upon the transcription factor Nanog for the maintenance of self-renewal capacity. Experiments conducted during the course of developing embodiments for the present invention determined that Nanog-regulated genes in head and neck squamous cell carcinoma (HNSCC) and NCCIT are dependent upon the coactivator p300, and that the KIX and CH1 domains of p300 are the specific subdomains that impact the regulation of Nanog-controlled genes. In addition, it was shown that overexpression of the CH1 domain in vivo blocked the formation of HNSCC tumors in a mouse model. It was also shown that small molecules that target p300 selectively inhibit the proliferation of CICs.

Accordingly, the present invention provides small compounds capable of modulating p300 and/or CBP-mediated transcription and related methods of therapeutic and research use. In addition, the present invention provides methods for treating conditions associated with aberrant p300 and/or CBP-mediated transcription with p300 and/or CBP-mediated transcription inhibitors.

In certain embodiments, the present invention provides methods for regulating CBP-mediated transcription of a gene of interest. The present invention is not limited to particular methods for regulating CBP-mediated transcription of a gene of interest. In some embodiments, the methods involve, for example, providing host cells expressing: CBP, a coactivator protein known to bind the KIX domain within CBP, and a gene of interest, wherein bind of the coactivator protein within the KIX domain is required for the CBP-mediated transcription of the gene of interest; and small molecules capable of binding within the KIX domain; and delivering to the host cells an effective amount of the small molecules such that expression of the gene of interest is modified. In some embodiments, the host cells are ex vivo, in vivo, in vitro, etc. In some embodiments, the host cells are cancer cells.

The methods are not limited to particular coactivator proteins. Examples of coactivator proteins involved with CBP-mediated transcription include, but are not limited to, MLL, Jun, Tat and Tax.

The methods are not limited to particular small molecules capable of binding within the KIX domain. In some embodiments, the small molecules are isoxazolidine compounds. In some embodiments, the small molecules are represented by the following formula:

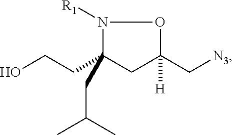

including salts, esters and prodrugs thereof, wherein R1 is a functional group facilitating binding within the KIX domain. The methods are not limited to a particular type or kind of R1. In some embodiments, R1 is configured to mimic at least a portion of amino acid residues 2840-2858 (ILPSDIMDFLVKNTP) (SEQ ID NO:1) within MLL. In some embodiments, R1 is configured to at least a portion of amino acid residues 47-66 (VLLKLASPELERLIIQSSN) (SEQ ID NO:2) within Jun. In some embodiments, R1 is configured to at least a portion of amino acid residues 1-24 (MEPVDPRLEPWKHPGSQPKT) (SEQ ID NO:3) within Tat. In some embodiments, R1 is configured to at least a portion of amino acid residues 76-95 (PSFPTQRTSKTLKVLPPIT) (SEQ ID NO: 4) within Tax. In some embodiments, R1 is selected from the group consisting of

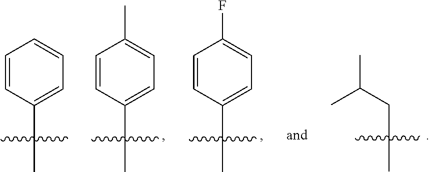

In some embodiments, the small molecule is selected from the group consisting of

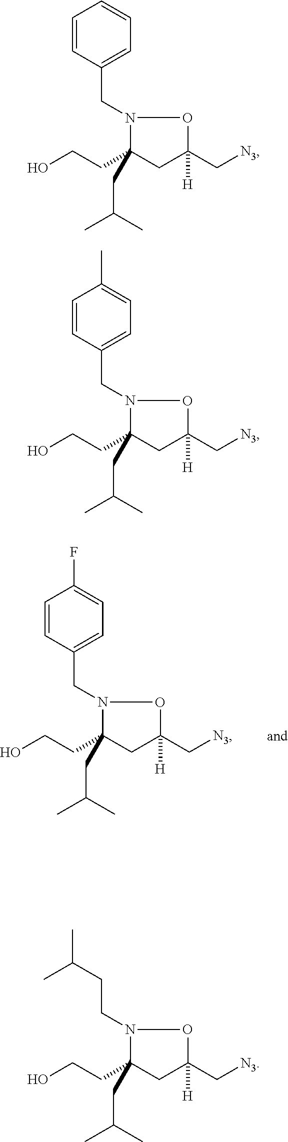

In certain embodiments, the present invention provides methods for regulating p300-mediated transcription of a gene of interest. The present invention is not limited to particular methods for regulating p300-mediated transcription. In some embodiments, the methods involve, for example, providing host cells expressing: p300, a coactivator protein known to bind the KIX and/or CH1 domains within p300, and a gene of interest, wherein binding of the coactivator protein within the KIX and/or CH1 domains is required for the CBP-mediated transcription of the gene of interest; and small molecules capable of binding within the KIX and/or CH1 domains; and delivering to the host cells an effective amount of the small molecules such that expression of the gene of interest is modified. In some embodiments, the host cells are ex vivo, in vivo, in vitro, etc. In some embodiments, the host cells are cancer cells. In some embodiments, the cancer cells are HNSCC cells. In some embodiments, the cancer cells are NCCIT cells.

The methods are not limited to a particular coactivator protein.

The methods are not limited to a particular small molecule that is capable of binding the KIX and/or CH1 domains of p300. In some embodiments, the small molecule is an isoxazolidine compound. In some embodiments, the isoxazolidine compounds are represented by the following formula:

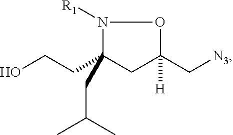

including salts, esters and prodrugs thereof, wherein R1 is a functional group facilitating binding within the KIX and/or CH1 domains. The methods are not limited to a particular structure for R1. In some embodiments, R1 is

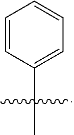 [SMILES: *c1ccccc1]

In some embodiments, the small molecule is

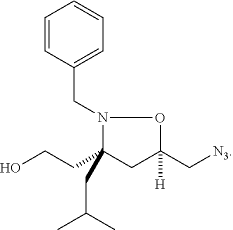 [SMILES: CC(C)C[C@]1(CCO)C[C@H](CN=[N+]=[N-])ON1Cc1ccccc1]

In certain embodiments, the present invention provides methods for treating a subject having a disorder having aberrant CBP related transcription. The present invention is not limited to particular methods for treating a subject having a disorder having aberrant CBP related transcription. In some embodiments, the methods involve, for example, administering to the subject a pharmaceutical composition comprising a CBP-mediated transcription inhibitor. In some embodiments, the subject is a human subject. In some embodiments, the disorder is cancer. The methods are not limited to particular type of cancer. In some embodiments, the cancer is leukemia. In some embodiments, the cancer is a solid tumor based cancer.

The methods are not limited to a particular type of CBP related transcription. In some embodiments, the CBP related transcription is MLL*CBP related transcription. In some embodiments, the CBP related transcription is Jun*CBP related transcription.

The methods are not limited to a particular type of CBP-mediated transcription inhibitor. In some embodiments, the CBP-mediated transcription inhibitor is an isoxazolidine compound. In some embodiments, the CBP-mediated transcription inhibitor is represented by the following formula:

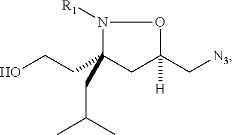

including salts, esters and prodrugs thereof, wherein R1 is a functional group facilitating binding within the KIX domain. The methods are not limited to a particular type or kind of R1. In some embodiments, R1 is configured to mimic at least a portion of amino acid residues 2840-2858 (ILPSDIMDFLVKNTP) (SEQ ID NO:1) within MLL. In some embodiments, R1 is configured to at least a portion of amino acid residues 47-66 (VLLKLASPELERLIIQSSN) (SEQ ID NO:2) within Jun. In some embodiments, R1 is configured to at least a portion of amino acid residues 1-24 (MEPVDPRLEPWKHPGSQPKT) (SEQ ID NO:3) within Tat. In some embodiments, R1 is configured to at least a portion of amino acid residues 76-95 (PSFPTQRTSKTLKVLPPIT) (SEQ ID NO: 4) within Tax. In some embodiments, R1 is selected from the group consisting of

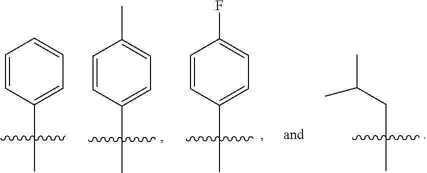

In some embodiments, the CBP-mediated transcription inhibitor is selected from the group consisting

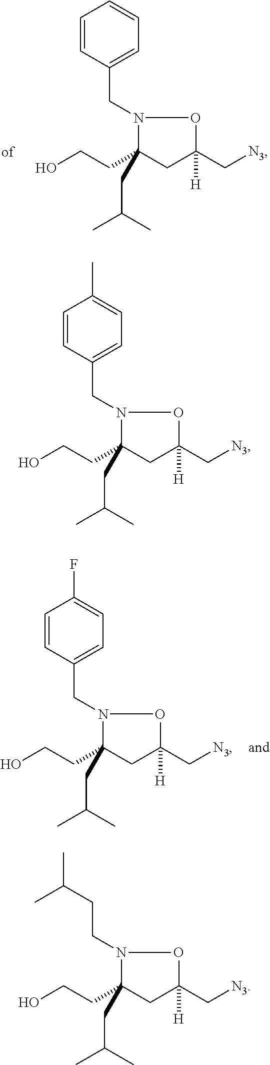

In some embodiments, the methods further involve, for example, co-administering to the subject effective amounts of one or more anti-cancer therapeutic agents.

In certain embodiments, the present invention provides methods for treating a subject having a disorder having aberrant p300 related transcription. The present invention is not limited to particular methods for treating a subject having aberrant p300 related transcription. In some embodiments, the methods involve, for example, administering to the subject a pharmaceutical composition comprising a p300-mediated transcription inhibitor. In some embodiments, the subject is a human subject. In some embodiments, the disorder is cancer. The methods are not limited to particular type of cancer. In some embodiments, the cancer is HNSCC. In some embodiments, the cancer comprises proliferating cancer-initiating cells.

The present invention is not limited to a particular type of p300 related transcription. In some embodiments, the p300 related transcription is Nanog*p300 related transcription.

The present invention is not limited to a particular type of p300-mediated transcription inhibitor. In some embodiments, the p300-mediated transcription inhibitor is an isoxazolidine compound. The methods are not limited to a particular type of isoxazolidine compound. In some embodiments, the isoxazolidine compounds is represented by the following formula:

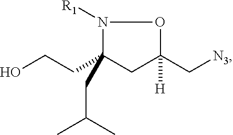

including salts, esters and prodrugs thereof, wherein R1 is a functional group facilitating binding within the KIX and/or CH1 domains. The methods are not limited to a particular type or kind of R1. In some embodiments, R1 is

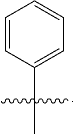 [SMILES: *c1ccccc1]

In some embodiments, the p300-mediated transcription inhibitor is

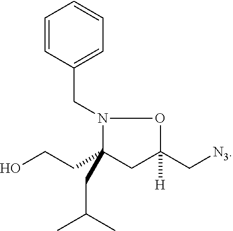

In some embodiments, the methods further comprise co-administering to the subject effective amounts of one or more anti-cancer therapeutic agents. The methods are not limited to a particular type or kind of anti-cancer therapeutic agent. In some embodiments, the anti-cancer therapeutic agent is salinomycin.

### BRIEF DESCRIPTION OF THE DRAWINGS

FIG. 1 shows (FIG. 1a) Transcriptional activators, minimally comprised of a DNA-binding domain (DBD) and a transcriptional activation domain (TAD), upregulate transcription by stimulating chromatin remodeling and facilitating the assembly of the RNA polymerase II holoenzyme at a promoter (see, e.g., Ansari, 2001). The TAD is primarily responsible for the direct binding interactions with the transcriptional machinery in order to accomplish this. Potential binding partners of TADs within the transcriptional machinery include enzymes that modify chromatin, the proteasome, and/or coactivators. Small or large molecule mimics of TADs can serve as competitive inhibitors of activators, thus down-regulating transcription. FIG. 1b) The two activator-binding sites within the KIX domain of CBP/p300. Highlighted in medium shade in the space-filling diagram of the KIX domain are the residues that experience the greatest chemical shift perturbation upon interacting with the TAD of MLL as measured by ¹H-¹⁵N-HSQC experiments (see, e.g., Goto, 2002); the TADs of Tat, Tax, and Jun occupy a similar binding site (see, e.g., Campbell, 2002; Vendel, 2004; Vendel, 2003). In darkest shade are the amino acids that change when interacting with Myb (see, e.g., De Guzman, 2006). Pymol figures were generated from 1kdx. (FIG. 1c) Isoxazolidine-based mimics of transcriptional activation domains. DBD=OxDex conjugated to an ethylene glycol linker (AEEA) (see, e.g., Minter, 2004; Buhrlage, 2005; Rowe, 2007).

FIG. 2 shows transcriptional activity of isoxazolidines 1a and 2a in the presence and absence of CBP. HeLa cells were transfected with 0.1 mL of a solution of Optimem containing a plasmid encoding the DBD of Ga14 fused to the ligand-binding domain of the glucocorticoid receptor (500 ng/mL), a firefly luciferase reporter plasmid (500 ng/mL) containing five Ga14 binding sites, and a Renilla transfection control (10 ng/mL); in addition, plasmids encoding CBP (100 ng/mL), a shRNA for CBP (100 ng/mL), or a scrambled shRNA sequence (250 ng/mL) were also transfected as indicated. Four hours post-transfection, 10 μM compound (1a and 2a) was added as a solution in DMSO such that the final concentration of DMSO was 1% (v/v). Luciferase output was measured 24 h after addition of compound; fold activation is a ratio of firefly and Renilla luminescence divided by the ratio of firefly and Renilla signal observed with DBD alone. Each value represents at least three individual experiments with the indicated error (standard deviation of the mean, SDOM). As illustrated, the activity of 2a is unaffected by CBP concentration. In addition, 1a activates transcription to equivalent levels when a scrambled shRNA is included. The scale of 0, 10, 20, 30, 40, 50, 60, 70, and 80 represents “fold activation.” “2a” is shown from first column from the left. “2a+CBP” is shown from second column from the left. “2a+CBP+shRNA” is shown from the third column from the left. “1a” is shown from fourth column from the left. “1a+scrambled shRNA” is shown from the fifth column from the left. “1a+CBP+shRNA” is shown from the sixth column from the left (the first column from the right).

FIG. 3 shows that CBP has no impact on baseline luciferase expression. HeLa cells were transfected with 0.1 mL of a solution of Optimem containing a plasmid containing a firefly luciferase reporter gene driven by a CMV promoter (500 ng/mL) and a Renilla control plasmid (10 ng/mL). Cells were also transfected with a plasmid encoding full-length CBP (100 ng/mL). Luciferase output was measured 24 h post-transfection revealing that CBP concentration has no impact on baseline luciferase expression. Fold activation is the ratio of firefly to Renilla signal divided by the ratio without CBP transfection. Each bar represents at least three experiments with the indicated error (SDOM). “CMV Luc” is shown as first column from the left. “CMV Luc+CBP” is shown as second column from the left (first column from the right).

FIG. 4 shows isoxazolidine-driven gene expression requires CBP. HeLa cells were transfected with a plasmid encoding the DBD of Ga14 fused to the ligand-binding domain of the glucocorticoid receptor (500 ng/mL), a firefly luciferase reporter plasmid containing five Ga14 binding sites (500 ng/mL), and a Renilla transfection control (10 ng/mL). Additional plasmids were transfected as indicated for CBP (100 ng/mL), CBP shRNA (100 ng/mL), and E1A (100 ng/mL) experiments. Four hours post-transfection, 10 μM 1a was added as a solution in DMSO such that the final concentration of DMSO was 1% (v/v). Luciferase output was measured 24 h after addition of compound; fold activation is a ratio of firefly and Renilla luminescence divided by the ratio of firefly and Renilla signal observed with DBD alone. Each value represents at least three individual experiments with the indicated error (standard deviation of the mean, SDOM). “1a” is shown as first column from the left. “1a+↑CBP” is shown as second column from the left. “1a+↓CBP” is shown as third column from the left. “1a+E1A” is shown as the fourth column form the left (first column from the right).

FIG. 5 shows inhibition of KIX-targeting TADs by 1b. (a) Isoxazolidine 1b decreased Jun and MLL minimal TAD-driven activation in a dose-dependent manner while not affecting ESX, an activator that is not known to bind the KIX domain (see, e.g., Kim, 2009). Cells were transfected with Firefly (500 ng/mL) and Renilla luciferase (10 ng/mL) plasmids in addition to plasmids encoding the DBD of Ga14 fused to the minimal activation domains of either MLL (250 ng/mL), Jun (100 ng/mL), or ESX (500 ng/mL, 0.1 mL of total transfection solution). Four hours post-transfection cells were treated with increasing concentration of 1b (0→50 μM) as a solution in DMSO such that the final DMSO concentration was 1% (v/v). Percent inhibition is the ratio of fold activation of Jun at each concentration of compound and the fold activation of Jun in a DMSO-treated control. Each bar represents at least three independent experiments with the indicated error (SDOM). For each concentration level of 1b shown in FIG. 5a, the first data column from the left is Jun, the middle column is MLL, and the third column is ESX. (b) MCF-7 cells were treated with 40 μM 1b (as a solution in DMSO such that the final concentration of DMSO was 1% v/v) for 24 h, at which time the cells were lysed and the lysates separated by SDS-PAGE. Western blots were performed using commercial antibodies against GAPDH, Jun, and cyclin D1.

FIG. 6 shows (a) Sequences of KIX-binding TADs. (b) Analogs of 1b tested for MLL and Jun inhibition. (c) Inhibition of Jun with isoxazolidine analogs Inhibition assays were carried out as described in FIG. 5a (SEQ ID NOs:1-4). For each concentration level shown in FIG. 6c, the compound data is shown (from the left) in the following order: 3, 4, 5, 6, 7, 8 and 9.

FIG. 7 shows a)¹H-¹⁵N HSQC spectrum of 200 μM ¹⁵N-His₆KIX (200 μM) alone or in the presences of 5 eq. of isoxazolidine 4. Samples were prepared in 9:1 H₂O: D₂O 10 mM phosphate buffer with 150 mM NaCl and <1% DMSO. b) An expanded portion of the ¹H-¹⁵N HSQC spectrum of 200 μM ¹⁵N-His₆KIX (200 μM) reveals several resonances that shift upon addition of 4 (FIG. 7B top). The amide chemical shifts that change upon addition of 4 were quantitated were quantified using the equation: Δδ=[Δδ(¹H)²+0.1Δδ(¹⁵N)²]^{1/2}, FIG. 7B bottom.

FIG. 8 shows cell viability of MCF-7 cells in the presence of increasing concentrations of isoxazolidine 1b and 1% DMSO. Viability data was acquired after 24 h using a cell-permeable metabolic dye, WST-1 (Roche). Viability data was normalized based on cell viability in the presence of 1% DMSO.

FIG. 9 shows HeLa cells transfected with firefly (500 ng/mL) and Renilla luciferase plasmids (10 ng/mL) in addition to plasmids encoding the DBD of Ga14 fused to the minimal activation domains of MLL (250 ng/mL). Four hours post-transfection cells were treated with increasing concentration of isoxazolidines 3-9 (0→10 μM) as a solution in DMSO such that the final DMSO concentration was 1% (v/v). Percent inhibition is the ratio of fold activation of MLL at each concentration of compound and the fold activation of MLL in a DMSO-treated control. Each bar represents at least three independent experiments with the indicated error (SDOM). For each concentration point, the compound data is shown (from the left) in the following order 3, 4, 5, 6, 7, 8 and 9.

FIG. 10 shows NMR spectra for various compounds.

FIGS. 11, 12 and 13 show impaired tumorsphere formation.

FIGS. 14A and 14B shows overexpression of the CH1 and KIX domains in HNSCC cells impacts Nanog transcriptional activity.

FIG. 15 shows expression of CH1 and KIX domains in combination more effectively impacts Nanog transcriptional activity.

FIG. 16 shows overexpression of the CH1 domain impacts A) Nanog expression, B) Nanog transcriptional activity, C) tumorsphere formation, and D) tumor formation in mice.

### DEFINITIONS

To facilitate an understanding of the invention, the following terms have the meanings defined below.

The term “host cell” or “cell” refers to any cell which is used in any of the methods of the present invention and may include prokaryotic cells, eukaryotic cells, yeast cells, bacterial cells, plant cells, animal cells, such as, reptilian cells, bird cells, fish cells, mammalian cells. Preferred cells include those derived from humans, dogs, cats, horses, cattle, sheep, pigs, llamas, gerbils, squirrels, goats, bears, chimpanzees, mice, rats, rabbits, etc. The term cells includes transgenic cells from cultures or from transgenic organisms. The cells may be from a specific tissue, body fluid, organ (e.g., brain tissue, nervous tissue, muscle tissue, retina tissue, kidney tissue, liver tissue, etc.), or any derivative fraction thereof. The term includes healthy cells, transgenic cells, cells affected by internal or exterior stimuli, cells suffering from a disease state or a disorder, cells undergoing transition (e.g., mitosis, meiosis, apoptosis, etc.), etc. The term also refers to cells in vivo or in vitro (e.g., the host cell may be located in a transgenic animal or in a human subject).

As used herein, the terms “host” and “subject” refer to any animal, including but not limited to, human and non-human animals (e.g. rodents, arthropods, insects (e.g., Diptera), fish (e.g., zebrafish), non-human primates, ovines, bovines, ruminants, lagomorphs, porcines, caprines, equines, canines, felines, ayes, etc.), that is studied, analyzed, tested, diagnosed or treated. As used herein, the terms “host” and “subject” are used interchangeably.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ non-translated sequences. The sequences that are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as activators. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The term “promoter region” refers to the 5′ flanking region of a gene and may contain regulatory sequences such as promoters and activators that control or influence the transcription of the gene. The 3′ flanking region may contain sequences that direct the termination of transcription, post-transcriptional cleavage and polyadenylation.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

As used herein, “expression” refers to the process by which nucleic acid is transcribed into mRNA and translated into peptides, polypeptides, or proteins. “Expression” may be characterized as follows: a cell is capable of synthesizing many proteins. At any given time, many proteins which the cell is capable of synthesizing are not being synthesized. When a particular polypeptide, coded for by a given gene, is being synthesized by the cell, that gene is said to be expressed. In order to be expressed, the DNA sequence coding for that particular polypeptide must be properly located with respect to the control region of the gene. The function of the control region is to permit the expression of the gene under its control. As used herein, the term “expression vector” includes vectors capable of expressing DNA or RNA fragments that are in operative linkage with regulatory sequences, such as promoter regions, that are capable of effecting expression of such DNA or RNA fragments. Thus, an expression vector refers to a recombinant DNA or RNA construct, such as a plasmid, a phage, recombinant virus or other vector that, upon introduction into an appropriate host cell, results in expression of the cloned DNA or RNA. Appropriate expression vectors are well known to those of skill in the art and include those that are replicable in eukaryotic cells and/or prokaryotic cells and those that remain episomal or may integrate into the host cell genome.

The term “gene transcription” as it is used herein means a process whereby one strand of a DNA molecule is used as a template for synthesis of a complementary RNA by RNA polymerase.

The term “pharmaceutical composition” refers to the combination of an active agent with a carrier, inert or active, making the composition especially suitable for diagnostic or therapeutic use in vivo or ex vivo.

The term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

The term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while sometime not in themselves pharmaceutically acceptable, may be employed in the preparation of salts useful as intermediates in obtaining the compounds of the invention and their pharmaceutically acceptable acid addition salts. Examples of bases include alkali metals (e.g., sodium) hydroxides, alkaline earth metals (e.g., magnesium), hydroxides, ammonia, and compounds of formula NW₄⁺, wherein W is C₁₋₄ alkyl, and the like. Examples of salts include, but are not limited to, acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “effective amount” refers to the amount of a compound (e.g., isoxazolidine compound) sufficient to effect beneficial or desired results. An effective amount can be administered in one or more administrations, applications or dosages and is not limited or intended to be limited to a particular formulation or administration route.

The term “second agent” refers to a therapeutic agent other than the isoxazolidine compounds in accordance with the present invention. In certain instances, the second agent is an anti-proliferative agent.

The term “co-administration” refers to the administration of at least two agent(s) (e.g., a compound of the present invention) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

The term “combination therapy” includes the administration of an isoxazolidine compound of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

### DETAILED DESCRIPTION

Experiments conducted during the course of developing embodiments for the present invention demonstrated that CBP plays an essential role in the ability of isoxazolidine transcriptional activation domains to activate transcription in cells. Consistent with this model, isoxazolidines were shown to be able to function as competitive inhibitors of the activators MLL and Jun, both of which utilize a binding interaction with KIX to up-regulate transcription. Further, it was shown that modification of the N2 side chain produced analogs with enhanced potency against Jun-mediated transcription, although increased cytotoxicity was also observed.

Very recently it has emerged that certain cancer cells exhibit self-renewal qualities that contribute both to their tendency to metastasize and their insensitivity to traditional therapies, contrary to the traditional models. These cells are referred to as ‘cancer stem cells’ or ‘cancer initiating cells’ (CI cells). In embryonic stem cells (ES cells), the master transcription factors Oct4, Sox2 and Nanog cooperatively up-regulate many of the genes required to maintain pluripotency while repressing pro-differentiation genes. In CI cells, the expression of Nanog and Oct4 is correlated with poorly differentiated tumors exhibiting self-renewal capacity and is also correlated with poor prognosis. However, CI cell circuitry is distinct from that of ES cells as Nanog plays the pre-eminent role in maintaining self-renewal capacity in CI cells; in contrast to ES cells, knockdown of Oct4 in CI cells has no observable effect on self-renewal or survival whereas knockdown of Nanog leads to loss of self-renewal, induction of differentiation programs, and apoptosis. Thus, molecules that directly regulate Nanog expression and function have the potential to transform understanding of the circuitry within cancer-initiating cells, the mechanisms by which currently intractable cancers are treated, and lead the way to next-generation therapeutic development. Nanog-regulated genes are dependent upon the p300. A unique characteristic of cancer-initiating cells (CICs or cancer stem cells) relative to embryonic or adult stem cells is a reliance upon the transcription factor Nanog for the maintenance of self-renewal capacity. Experiments conducted during the course of developing embodiments for the present invention determined that Nanog-regulated genes in head and neck squamous cell carcinoma (HNSCC) and NCCIT are dependent upon the coactivator p300, and that the KIX and CH1 domains of p300 are the specific subdomains that impact the regulation of Nanog-controlled genes. In addition, it was shown that overexpression of the CH1 domain in vivo blocked the formation of HNSCC tumors in a mouse model. It was also shown that small molecules that target p300 selectively inhibit the proliferation of CICs.

Accordingly, the present invention provides small molecules (e.g., compounds) capable of inhibiting p300 and/or CBP-mediated transcription and their therapeutic and/or research uses. Exemplary compositions and methods of the present invention are described in more detail in the following sections: I. p300 and CBP-Mediated Transcription Modulators; II. Methods for Identifying p300 and CBP-Mediated Transcription Modulators; III. Methods for Regulating p300 and CBP-Mediated Gene Transcription; IV. Therapeutics and Research Applications; V. Pharmaceutical Compositions; and VI. Other Embodiments.

### I. p300 and CBP-Mediated Transcription Modulators

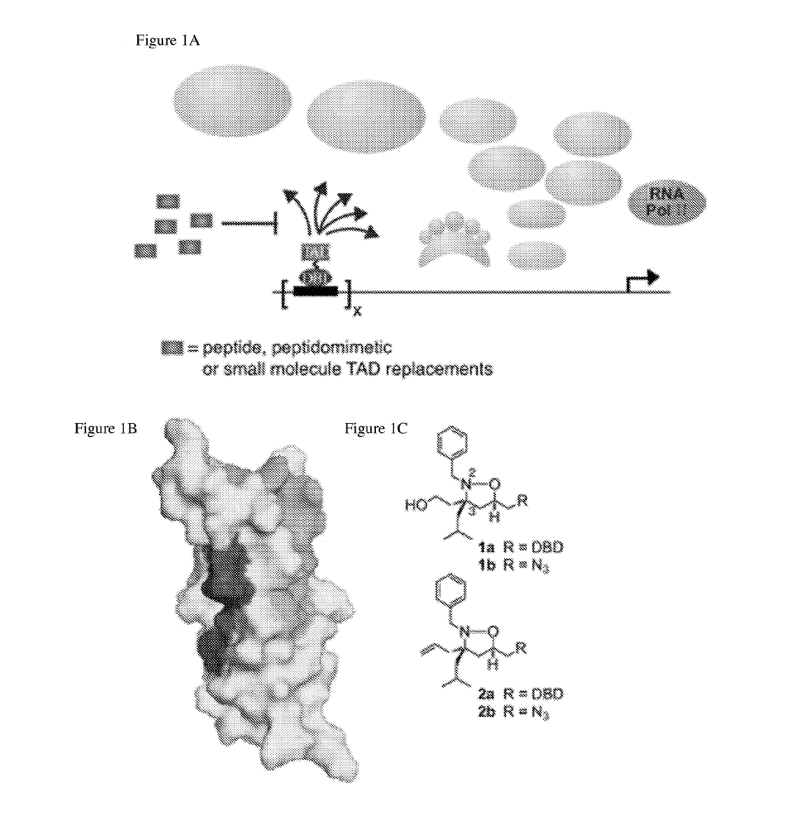

Transcription factors, often characterized as undruggable, are nonetheless emerging as potentially attractive therapeutic targets due to their fundamental role in human disease (see, e.g., Verdine, 2007; Pandolfi, 2001; Darnell, 2002; Koh, 2007; Koehler, 2010; Lee, 2010). One class of transcription factors, transcriptional activators, plays a key regulatory role by binding to DNA and assembling the transcriptional machinery at a particular gene, thus stimulating gene expression. Miscued transcription caused by malfunctioning activators has been implicated in the onset of many cancers and this has spurred widespread interest in the development of small and large molecules that directly target misregulated genes (see, e.g., Pandolfi, 2001; Darnell, 2002; Koh, 2007; Koehler, 2010). The development of small molecule transcriptional activation domain (TAD) mimics is an advantageous strategy to modulate transcription because, similar to natural TADs, these molecules can function as activators when tethered to a DNA-binding domain (DBD) and as inhibitors when not localized to DNA (FIG. 1a) (see, e.g., Lee, 2010). TAD mimics are thus promising candidates for modulating gene transcription, for use as mechanistic probes, and as transcription-targeted therapeutics (see, e.g., Lee, 2010).

Like their natural counterparts, small molecule TADs must interact with coactivators in the transcriptional machinery in order to function. This presents a formidable challenge since the number of putative coactivator targets of activators is large, with few validated as physiologically relevant (see, e.g., Mapp, 2007).

One target of importance is the CREB binding protein (CBP), a large (265 kDa) multidomain coactivator and histone acetyl transferase (see, e.g., Vo, 2001; Goodman, 2000). CBP integrates transcriptional signals from >100 transcription factors and is an essential protein for cell growth and development. In contrast to most coactivators, the individual domains of CBP have proven amenable to structural characterization and, concomitantly, have been attractive targets for the development of small and large molecule transcriptional regulators (see, e.g., Kung, 2004; Liu, 2005; Buhrlage, 2009; Cebrat, 2003; Best, 2004; Henchey, 2010; Li, 2009). A domain that has attracted much attention in this regard is the KIX domain, an 87-residue module whose solution structure has been elucidated (see, e.g., Radhakrishnan, 1997). Despite its relatively small size, the KIX domain contains at least two activator binding sites, the CREB/Myb site and the MLL/Jun/Tat/Tax site (FIG. 1b) (see, e.g., Radhakrishnan, 1999; Goto, 2002; Zor, 2004; Campbell, 2002; Vendel, 2004; Vendel, 2003). Screening against the KIX domain has led to the discovery of both peptides and peptidomimetic TADs that function well in cells (see, e.g., Liu, 2005; Frangioni, 2000); derivatives of these peptides have been useful tools for defining characteristics of activator binding sites (see, e.g., Rowe, 2008). Not just useful for creating activators, a small molecule screen against the KIX domain has also yielded naphthol AS-E phosphate and related derivatives that competitively inhibit the KIX CREB binding interaction (see, e.g., Best, 2004; Li, 2009). Complementary to genetic strategies, compounds that inhibit KIX-binding activators will be useful tools in dissecting the role of the KIX domain in physiological processes such as cell differentiation and hematopoiesis (see, e.g., Iyer, 2004; Kasper, 2006; Iyer, 2007; Kimbrel, 2009). Furthermore, both MLL and Jun have been implicated in several cancers, including leukemias and solid tumors (see, e.g., Kinoshita, 2003; Hanson, 1997; Li, 2000; Ayton, 2003; Leaner, 2003; Wisdom, 1999); thus compounds that block these proteins from forming key interactions with CBP may prove useful in the development of transcription targeted therapeutics (see, e.g., De Guzman, 2006; Verdine, 2007; Pandolfi, 2001; Darnell, 2002; Koh, 2007; Koehler, 2010; Lee, 2010; Mapp, 2007; Vo, 2001; Goodman, 2000).

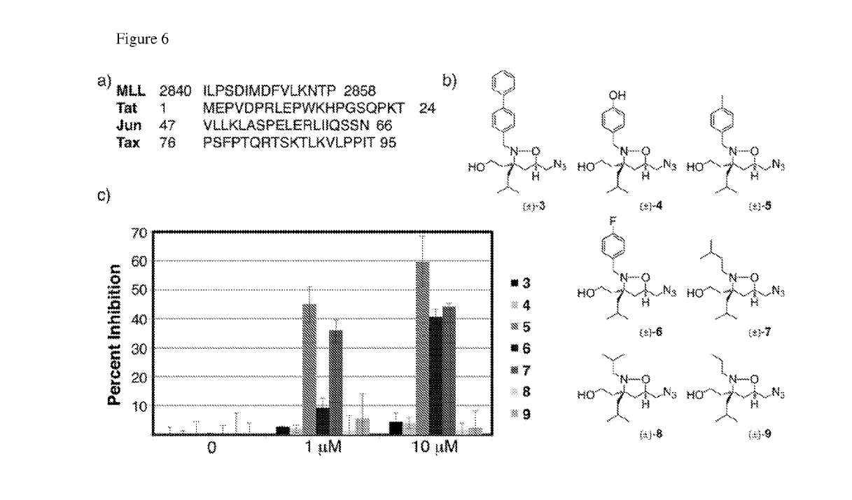

Several isoxazolidine-based TADs have been identified through a top-down discovery strategy that reconstitute the function of a natural activator when localized to a promoter (e.g. isoxazolidine 1a, FIG. 1c) (see, e.g., Minter, 2004; Buhrlage, 2005; Rowe, 2007). Subsequent in vitro binding studies revealed that one target of these small molecules is the KIX domain of CBP, more specifically the MLL/Jun/Tat/Tax site within that domain (FIG. 1b) (see, e.g., Buhrlage, 2009). Experiments conducted during the course of developing embodiments for the present invention determined that the isoxazolidine*CBP interaction is required for cellular activity. Consistent with this model, in the absence of DNA binding, isoxazolidine 1 (see, FIG. 1c) was shown to be able to competitively inhibit transcription mediated by the KIX-dependent activators MLL and c-Jun. Based on natural peptide ligands for KIX, alteration of the aromatic side chain of isoxazolidine 1 led to the identification of two molecules with enhanced potency (isoxazolidine 5, isoxazolidine 6, isoxazolidine 7; FIG. 6).

Nanog-regulated genes are dependent upon the p300. A unique characteristic of cancer-initiating cells (CICs or cancer stem cells) relative to embryonic or adult stem cells is a reliance upon the transcription factor Nanog for the maintenance of self-renewal capacity. Experiments conducted during the course of developing embodiments for the present invention determined that Nanog-regulated genes in head and neck squamous cell carcinoma (HNSCC) and human teratocarcinoma cells (NCCIT) are dependent upon the coactivator p300, and that the KIX and CH1 domains of p300 are the specific subdomains that impact the regulation of Nanog-controlled genes. In addition, it was shown that overexpression of the CH1 domain in vivo blocked the formation of HNSCC tumors in a mouse model. It was also shown that small molecules that target p300 selectively inhibit the proliferation of CICs.

Accordingly, the present invention provides p300 and CBP-mediated transcription modulators. The present invention is not limited to p300-mediated transcription modulators or CBP-mediated transcription modulators. In some embodiments the transcription modulators are specific to either p300-mediated transcription or CBP-mediated transcription. In some embodiments, the transcription modulators are specific for both p300-mediated transcription and CBP-mediated transcription. The present invention is not limited to particular types or kinds of p300 and CBP-mediated transcription modulators. In some embodiments, the p300 and CBP-mediated transcription modulators are p300 and CBP-mediated transcription inhibitors. In some embodiments, the p300 and CBP-mediated transcription modulators are p300 and CBP-mediated transcription activators.

The present invention is not limited to particular types of p300 and CBP-mediated transcription inhibitors or activators. In some embodiments, the prevention invention provides small molecules capable of inhibiting or activating p300-mediated transcription and/or CBP-mediated transcription. The present invention is not limited to particular types of small molecules capable of inhibiting or activating p300-mediated transcription and/or CBP-mediated transcription. In some embodiments, such small molecules are configured to bind specific domains within p300 and/or CBP. The present invention is not limited to particular domains within p300 and/or CBP for which such small molecules bind. In some embodiments, the small molecules are configured to bind the KIX and/or CH1 domain within p300. The small molecules are not limited to binding a particular region within the KIX or CH1 domains of p300. In some embodiments, the small molecules are configured to bind the KIX domain within CBP. The small molecules are not limited to binding a particular region within the KIX domain of CBP. In some embodiments, the small molecules are configured to bind the MLL/Jun/Tat/Tax region within the KIX domain of CBP.

In some embodiments, by binding with either the KIX domain within CBP or the KIX and/or CH1 domains within p300, the small molecules attenuate the binding of coactivator proteins known to bind such regions. For example, experiments conducted during the course of developing embodiments for the present invention demonstrated that small molecules that bind the MLL/Jun/Tat/Tax region within the KIX domain of CBP attenuate CBP*MLL binding within that region, thereby inhibiting CBP-mediated transcription requiring such interaction with MLL. Such experiments additionally demonstrated that small molecules that bind the MLL/Jun/Tat/Tax region within the KIX domain of CBP attenuate CBP*Jun binding within that region, thereby inhibiting CBP-mediated transcription requiring such interaction with Jun. Such experiments additionally demonstrated that small molecules that bind the KIX and/or CH1domains of p300 attenuate p300*Nanog binding within that region, thereby inhibiting p300-mediated transcription requiring such interaction with Nanog (e.g., thereby inhibiting CIC proliferation). It is contemplated that small molecules that bind the MLL/Jun/Tat/Tax region within the KIX domain of CBP attenuate CBP*Tat binding within that region, thereby inhibiting CBP-mediated transcription requiring such interaction with Tat. It is contemplated that small molecules that bind the MLL/Jun/Tat/Tax region within the KIX domain of CBP attenuate CBP*Tax binding within that region, thereby inhibiting CBP-mediated transcription requiring such interaction with Tax.

The small molecules are not limited to a particular manner or structures that facilitate such interacting (e.g., binding) with CBP (e.g., binding within the KIX domain (MLL/Jun/Tat/Tax region). In some embodiments, the structure of the small molecule is such that it is able to mimic a coactivator protein known to bind the particular region within CBP. For example, in some embodiments, the small molecule is configured to mimic the amino acid portion of MLL known to bind the KIX domain of CBP (e.g., at least a portion of amino acid residues 2840-2858 (ILPSDIMDFLVKNTP) (SEQ ID NO:1) within MLL) (e.g., (thereby facilitating binding of the small molecule within the respective region). In some embodiments, the small molecule is configured to mimic the amino acid portion of Jun known to bind the KIX domain of CBP (e.g., at least a portion of amino acid residues 47-66 (VLLKLASPELERLIIQSSN) (SEQ ID NO:2) within Jun) (thereby facilitating binding of the small molecule within the respective region). In some embodiments, the small molecule is configured to mimic the amino acid portion of Tat known to bind the KIX domain of CBP (e.g., at least a portion of amino acid residues 1-24 (MEPVDPRLEPWKHPGSQPKT) (SEQ ID NO:3) within Tat) (thereby facilitating binding of the small molecule within the respective region). In some embodiments, the small molecule is configured to mimic the amino acid portion of Tax known to bind the KIX domain of CBP (e.g., at least a portion of amino acid residues 76-95 (PSFPTQRTSKTLKVLPPIT) (SEQ ID NO: 4) within Tax) (thereby facilitating binding of the small molecule within the respective region).

The small molecules are not limited to a particular manner or structure that facilitate such interacting (e.g., binding) with the KIX and/or CH1 domains of p300 (e.g., binding within the CH1 domain). In some embodiments, the small molecule is configured to mimic the amino acid portion of Nanog known to bind the KIX and/or CH1 domains of p300 (thereby facilitating binding of the small molecule within the respective region).

The present invention is not limited to particular types of small molecules that inhibit p300 and/or CBP-mediated transcription by binding with either the KIX domain of CBP or the KIX and/or CH1 domains of p300 (e.g., thereby attenuating binding of MLL, Jun, Tat and/or Tax within the KIX domain of CBP) (e.g., thereby attenuating binding of Nanog within the KIX and/or CH1 domains of p300). In some embodiments, such small molecules are isoxazolidine based compounds (see, e.g., U.S. Pat. No. 7,786,310). In some embodiments, the isoxazolidine based compounds comprise one or more functional groups facilitating interaction (e.g., binding) within the KIX domain of CBP (e.g., the MLL/Jun/Tat/Tax region of the KIX domain within p300/CBP) or p300. In some embodiments, the isoxazolidine based compounds comprise one or more functional groups facilitating interaction (e.g., binding) within the CH1 domain of p300.

In some embodiments, isoxazoline compounds having the following formula is provided:

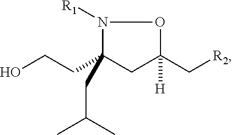

including salts, esters and prodrugs thereof.

In some embodiments, R1 is a functional group facilitating interaction (e.g., binding) within the KIX domain of CBP (e.g., the MLL/Jun/Tat/Tax region of the KIX domain within CBP) or p300. In some embodiments, R1 is configured to mimic the amino acid portion of MLL known to bind the KIX domain of CBP (e.g., at least a portion of amino acid residues 2840-2858 (ILPSDIMDFLVKNTP) (SEQ ID NO:1) within MLL) (e.g., thereby facilitating binding of the isoxazolidine compound within the respective region). In some embodiments, R1 is configured to mimic the amino acid portion of Jun known to bind the KIX domain of CBP (e.g., at least a portion of amino acid residues 47-66 (VLLKLASPELERLIIQSSN) (SEQ ID NO:2) within Jun) (thereby facilitating binding of the isoxazolidine compound within the respective region). In some embodiments, R1 is configured to mimic the amino acid portion of Tat known to bind the KIX domain of CBP (e.g., at least a portion of amino acid residues 1-24 (MEPVDPRLEPWKHPGSQPKT) (SEQ ID NO:3) within Tat) (thereby facilitating binding of the isoxazolidine compound within the respective region). In some embodiments, R1 is configured to mimic the amino acid portion of Tax known to bind the KIX domain of CBP (e.g., at least a portion of amino acid residues 76-95 (PSFPTQRTSKTLKVLPPIT) (SEQ ID NO: 4) within Tax) (thereby facilitating binding of the isoxazolidine compound within the respective region).

Experiments conducted during the course of developing embodiments for the present invention determined that a phenyl group at R1 facilitated (e.g.,

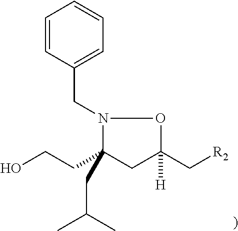

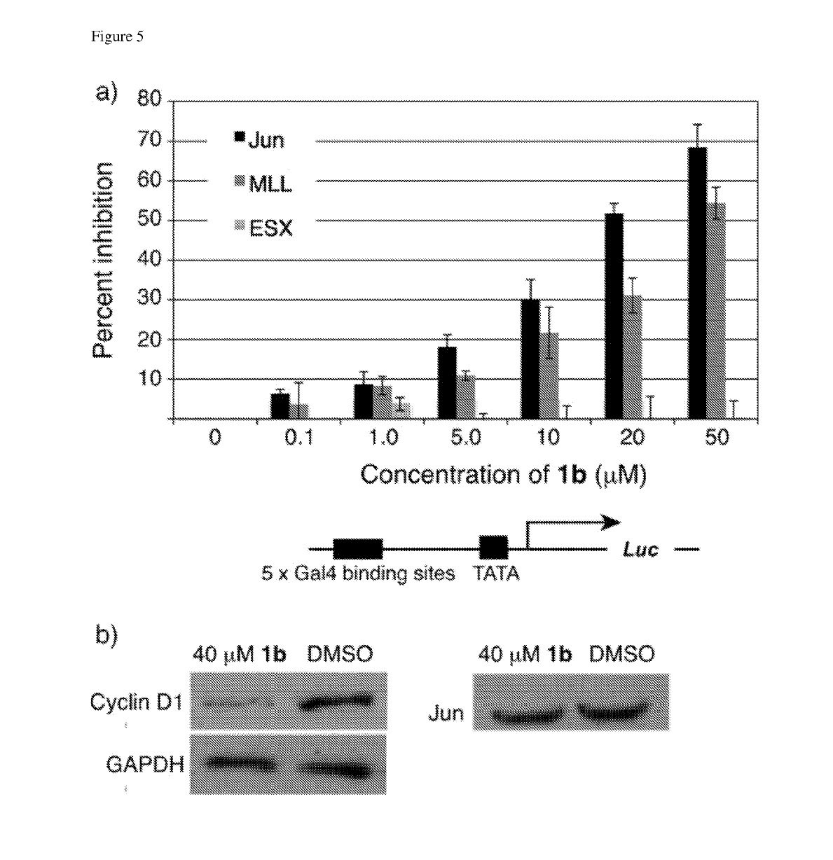

binding within the KIX domain of CBP and the KIX domain of p300 and, upon such binding, competitively inhibited binding between CBP*MLL and CBP*Jun (see FIG. 5). Such experiments additionally demonstrated the following groups for R1

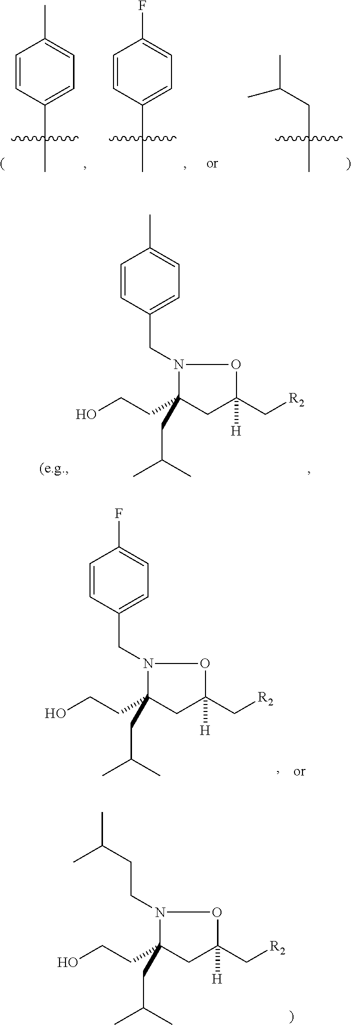

facilitated binding within the KIX domains of CBP and p300 and, upon such binding, competitively inhibited binding between CBP*Jun (see FIG. 6). As such, in some embodiments, the following isoxazolidine compounds are provided as facilitating interaction (e.g., binding) within the KIX domain of CBP (e.g., the MLL/Jun/Tat/Tax region of the KIX domain within CBP) and/or p300:

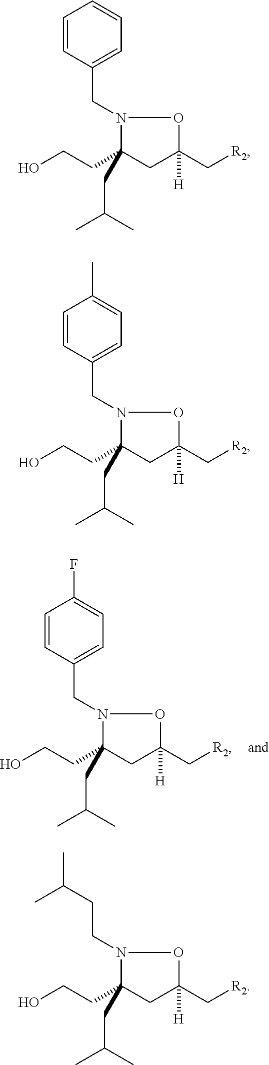

In some embodiments, R1 is a functional group facilitating interaction (e.g., binding) within the CH1 domain of p300. In some embodiments, R1 is configured to mimic at least a portion of the amino acid portion of Nanog known to bind the CH1 domain of p300 (thereby facilitating binding of the small molecule within the respective region).

In some embodiments, R2 is a functional group inhibiting binding to a DNA binding domain for a gene of interest. For example, in embodiments where the isoxazolidine compound is functioning as a competitive inhibitor for molecules that bind the KIX domain of CBP or p300 and/or the CH1 domain of p300, R2 is any functional group that would inhibit binding with a DNA binding domain of a gene of interest. In such embodiments, R2 is N₃. In some embodiments, R2 is a DNA binding domain for any gene of interest.

The foregoing p300 and/or CBP-mediated transcriptional modulators (e.g., small molecules capable of binding within the KIX domain of CBP) (e.g., small molecules capable of binding within the KIX and/or CH1 domains of p300) can be present in pharmaceutical compositions comprising a compound described herein and a pharmaceutically acceptable carrier. In certain embodiments, the pharmaceutical composition further comprises a therapeutic agent. For example, in some embodiments, the therapeutic agent is directed toward treating disorders having aberrant p300 and/or CBP-mediated transcriptional activity (e.g., leukemia, solid tumors, various cancers). Examples of such therapeutics include any type/kind of cancer therapeutic. In addition, in some embodiments, the therapeutic agent is directed toward inhibiting cancer initiating cells and related types of cancer (e.g., solid tumors of the brain, breast, colon, ovary, pancreas, prostate, melanoma and multiple melanoma; see, e.g., Singh, 2003; Al-Hajj, 2003; O'Brien, 2007; Zhang, 2008; Li, 2007; Maitland, 2008; Lang, 2009; Schatton, 2008; Boiko, 2010; Schmidt, 2011; Civenni, 2011; Matsui, 2004; Matsui, 2008). In some embodiments, the therapeutic agent targets cancer initiating cell related pathways (e.g., therapeutic agents that target pathways involved in cancer initiating cells (e.g., Bmi-1 (see, e.g., Park, 2003; Hemmati, 2003), Notch (see, e.g., Dievart, 1999), sonic hedgehog and Wnt). In some embodiments, the therapeutic agent is salinomycin (see, e.g., Gupta, 2009).

### II. Methods for Identifying p300 and/or CBP-Mediated Transcription Modulators

The present invention provides methods for identifying small molecules capable of modulating (e.g., inhibiting, activating) p300 and/or CBP-mediated transcription. The methods are not limited to a particular methods.

In some embodiments, algorithms such as TFSEARCH or JASPAR are used to identify putative p300 and/or CBP binding sites. Upon identification of such putative p300 and/or CBP binding sites, small molecules can be constructed as potential p300 and/or CBP-mediated transcription modulators.

### III. Methods for Regulating p300 and/or CBP-Mediated Gene Expression

The present invention provides methods for regulating expression of a gene known to be regulated by p300 and/or CBP. For example, the present invention provides methods for regulating p300-mediated transcription comprising providing, for example, host cells (e.g., in vivo, ex vivo, in vitro) (e.g., cancer initiating cells) expressing Nanog, p300 and a p300-mediated transcription inhibitors (e.g., small molecules capable of binding within the KIX and/or CH1 domains of p300), and delivering to the host cells an effective amount of the p300-mediated transcription inhibitors. Similarly, in some embodiments, the present invention provides methods for regulating CBP-mediated transcription comprising providing, for example, host cells (e.g., in vivo, ex vivo, in vitro) expressing MLL, Tat, Tax and/or Jun, CBP and a CBP-mediated transcription inhibitors (e.g., small molecules capable of binding within the KIX domain of CBP), and delivering to the host cells an effective amount of the CBP-mediated transcription inhibitors.

### IV. Therapeutics and Research Applications

The present invention provides methods for regulating p300 and/or CBP-mediated transcription of a gene of interest in a subject for the purpose of, for example, analyzing the effect of a p300 and/or CBP-mediated transcription modulator, modulating transcription to assist with therapy (e.g., co-administered with existing therapies) or as a standalone therapy, comprising: providing a subject and a p300 and/or CBP-mediated transcription modulator and delivering to the subject an effective amount of the p300 and/or CBP-mediated transcription modulator such that expression of the gene of interest is modified (e.g., inhibited, enhanced).

Experiments conducted during the course of developing embodiments for the present invention determined that particular isoxazolidine compounds were able to bind the KIX domain of CBP thereby attenuating binding between CBP*MLL and CBP*Jun. Accordingly, the present invention provides methods for treating diseased cells, tissues, organs, or pathological conditions and/or disease states associated with MLL and/or Jun activity (e.g., cancers for which MLL and/or Jun are implicated; see, e.g., Kinoshita, 2003; Hanson, 1997; Li, 2000; Ayton, 2003; Leaner, 2003; Wisdom, 1999) through administration of compounds (e.g., isoxazolidine compounds; isoxazolines 1, 5, 6, and 7 from FIGS. 1 and 6) that block MLL and/or Jun from forming key interactions with CBP to subjects (e.g., human patients) suffering from such diseases/disorders. In some embodiments, additional therapeutic agents are co-administered with such isoxazolidine compounds (e.g., therapeutic agents directed toward treating cancers). In some embodiments, the additional therapeutics include any type or kind of cancer therapeutic.

Experiments conducted during the course of developing embodiments for the present invention determined that small molecules that target the KIX and/or CH1 domains of p300 attenuate Nanog-regulated genes and, in particular, inhibit the proliferation of cancer-initiating cells (e.g., cancer stem cells) (e.g., HNSCC cells) (e.g., NCCIT cells). As such, the present invention provides methods for treating a subject (e.g., human subject) suffering from a cancer associated with proliferating cancer-initiating cells through administering to the subject compounds (e.g., isoxazolidine compounds; isoxazolines 1, 5, 6, and 7 from FIGS. 1 and 6) that target the KIX and/or CH1 domains of p300, thereby attenuating Nanog-regulated gene expression and inhibiting proliferation of the cancer-initiating cells. Examples of cancers associated with cancer-initiating cells include, but are not limited to, solid tumors of the brain, breast, colon, ovary, pancreas, prostate, melanoma and multiple melanoma (see, e.g., Singh, 2003; Al-Hajj, 2003; O'Brien, 2007; Zhang, 2008; Li, 2007; Maitland, 2008; Lang, 2009; Schatton, 2008; Boiko, 2010; Schmidt, 2011; Civenni, 2011; Matsui, 2004; Matsui, 2008). In addition, in some embodiments, additional therapeutic agents are co-administered with such compounds that target the KIX and/or CH1 domains of p300. Examples of such additional therapeutic agents include, but are not limited to, therapeutic agents that target cancer initiating cell related pathways (e.g., therapeutic agents that target pathways involved in cancer initiating cells (e.g., Bmi-1 (see, e.g., Park, 2003; Hemmati, 2003), Notch (see, e.g., Dievart, 1999), sonic hedgehog and Wnt). In some embodiments, the therapeutic agent is salinomycin (see, e.g., Gupta, 2009). In some embodiment, the therapeutic agent is any cancer therapeutic.

Examples of cancer therapeutics include, but are not limited to, Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N, N′-Bis(2-chloroethyl)-N-nitrosourea (BCNU); N-(2-chloroethyl)-N′-cyclohex-yl-N-nitrosourea (CCNU); N-(2-chloroethyl)-N-(trans-4-methylcyclohexyl-N-nitrosourea (MeCCNU); N-(2-chloroethyl)-N-(diethyl)ethylphosphonate-N-nit-rosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); and 2-chlorodeoxyadenosine (2-Cda). Other anti-cancer agents include, but are not limited to, Antiproliferative agents (e.g., Piritrexim Isothionate), Antiprostatic hypertrophy agent (e.g., Sitogluside), Benign prostatic hyperplasia therapy agents (e.g., Tamsulosin Hydrochloride), Prostate growth inhibitor agents (e.g., Pentomone), and Radioactive agents: Fibrinogen 1 125; Fludeoxyglucose F 18; Fluorodopa F 18; Insulin I 125; Insulin I 131; Iobenguane I 123; Iodipamide Sodium I 131; Iodoantipyrine I 131; Iodocholesterol I 131; Iodohippurate Sodium I 123; Iodohippurate Sodium I 125; Iodohippurate Sodium I 131; Iodopyracet I 125; Iodopyracet I 131; Iofetamine Hydrochloride I 123; Iomethin I 125; Iomethin I 131; Iothalamate Sodium I 125; Iothalamate Sodium I 131; Iotyrosine I 131; Liothyronine I 125; Liothyronine I 131; Merisoprol Acetate Hg 197; Merisoprol Acetate Hg 203; Merisoprol Hg 197; Selenomethionine Se 75; Technetium Tc 99m Antimony Trisulfide Colloid; Technetium Tc 99m Bicisate; Technetium Tc 99m Disofenin; Technetium Tc 99m Etidronate; Technetium Tc 99m Exametazime; Technetium Tc 99m Furifosmin; Technetium Tc 99m Gluceptate; Technetium Tc 99m Lidofenin; Technetium Tc 99m Mebrofenin; Technetium Tc 99m Medronate; Technetium Tc 99m Medronate Disodium; Technetium Tc 99m Mertiatide; Technetium Tc 99m Oxidronate; Technetium Tc 99m Pentetate; Technetium Tc 99m Pentetate Calcium Trisodium; Technetium Tc 99m Sestamibi; Technetium Tc 99m Siboroxime; Technetium Tc 99m Succimer; Technetium Tc 99m sulfur Colloid; Technetium Tc 99m Teboroxime; Technetium Tc 99m Tetrofosmin; Technetium Tc 99m Tiatide; Thyroxine I 125; Thyroxine I 131; Tolpovidone I 131; Triolein I 125; and Triolein I 131).

Additional anti-cancer agents include, but are not limited to anti-cancer Supplementary Potentiating Agents: Tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and Multiple Drug Resistance reducing agents such as Cremaphor EL. Still other anticancer agents include, but are not limited to, annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; Hydroxyurea; Docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-C1-2′deoxyadenosine; Fludarabine-PO₄; mitoxantrone; mitozolomide; Pentostatin; and Tomudex. One particularly preferred class of anticancer agents are taxanes (e.g., paclitaxel and docetaxel). Another important category of anticancer agent is annonaceous acetogenin.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

In some embodiments, methods provided herein comprise administering one or more p300 and/or CBP-mediated transcription inhibitors with radiation therapy. The methods provided herein are not limited by the types, amounts, or delivery and administration systems used to deliver the therapeutic dose of radiation to an animal. For example, the animal may receive photon radiotherapy, particle beam radiation therapy, other types of radiotherapies, and combinations thereof. In some embodiments, the radiation is delivered to the animal using a linear accelerator. In still other embodiments, the radiation is delivered using a gamma knife.

The source of radiation can be external or internal to the animal. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by animals. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The animal may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to an animal, so long as the dose of radiation is tolerated by the animal without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

### V. Pharmaceutical Compositions, Formulations, and Exemplary Administration Routes and Dosing Considerations

Exemplary embodiments of various contemplated medicaments and pharmaceutical compositions are provided below.

### A. Preparing Pharmaceutical Formulations

The p300 and/or CBP-mediated transcription modulators (e.g., small molecules capable of binding within the KIX and/or CH1 domains of p300) (e.g., small molecules capable of binding within the KIX domain of CBP) are useful in the preparation of pharmaceutical formulation, also synonymously referred to herein as “medicaments,” to treat a variety of conditions (e.g., cancers associated with cancer-initiating cells) (e.g., cancers for which MLL and Jun are implicated). The methods and techniques for preparing medicaments of p300 and/or CBP-mediated transcription inhibitors are well-known in the art. Exemplary pharmaceutical formulations and routes of delivery are described below.

One of skill in the art will appreciate that any one or more of the p300 and/or CBP-mediated transcription inhibitors described herein are prepared by applying standard pharmaceutical manufacturing procedures. Such pharmaceutical formulations can be delivered to the subject by using delivery methods that are well-known in the pharmaceutical arts.

### B. Exemplary Pharmaceutical Compositions and Formulation

In some embodiments of the present invention, the compositions are administered alone, while in some other embodiments, the compositions are preferably present in a pharmaceutical formulation comprising at least one active ingredient/agent, as defined above, together with a solid support or alternatively, together with one or more pharmaceutically acceptable carriers and optionally other therapeutic agents. Each carrier must be “acceptable” in the sense that it is compatible with the other ingredients of the formulation and not injurious to the subject.

Contemplated formulations include those suitable oral, rectal, nasal, topical (including transdermal, buccal and sublingual), vaginal, parenteral (including subcutaneous, intramuscular, intravenous and intradermal) and pulmonary administration. In some embodiments, formulations are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present invention suitable for oral administration may be presented as discrete units such as capsules, cachets or tablets, wherein each preferably contains a predetermined amount of the active ingredient; as a powder or granules; as a solution or suspension in an aqueous or non-aqueous liquid; or as an oil-in-water liquid emulsion or a water-in-oil liquid emulsion. In other embodiments, the active ingredient is presented as a bolus, electuary, or paste, etc.

In some embodiments, tablets comprise at least one active ingredient and optionally one or more accessory agents/carriers are made by compressing or molding the respective agents. In some embodiments, compressed tablets are prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets are made by molding in a suitable machine a mixture of the powdered compound (e.g., active ingredient) moistened with an inert liquid diluent. Tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention are optionally formulated as ointments, creams, suspensions, lotions, powders, solutions, pastes, gels, sprays, aerosols or oils. In alternatively embodiments, topical formulations comprise patches or dressings such as a bandage or adhesive plasters impregnated with active ingredient(s), and optionally one or more excipients or diluents. In some embodiments, the topical formulations include a compound(s) that enhances absorption or penetration of the active agent(s) through the skin or other affected areas. Examples of such dermal penetration activators include dimethylsulfoxide (DMSO) and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof.

In some embodiments, oily phase emulsions of this invention are constituted from known ingredients in a known manner. This phase typically comprises a lone emulsifier (otherwise known as an emulgent), it is also desirable in some embodiments for this phase to further comprises a mixture of at least one emulsifier with a fat or an oil or with both a fat and an oil.

Preferably, a hydrophilic emulsifier is included together with a lipophilic emulsifier so as to act as a stabilizer. It some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus creams should preferably be a non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with suitable base comprising, for example, cocoa butter or a salicylate. Likewise, those for vaginal administration may be presented as pessaries, creams, gels, pastes, foams or spray formulations containing in addition to the agent, such carriers as are known in the art to be appropriate.

Formulations suitable for nasal administration, wherein the carrier is a solid, include coarse powders having a particle size, for example, in the range of about 20 to about 500 microns which are administered in the manner in which snuff is taken, i.e., by rapid inhalation (e.g., forced) through the nasal passage from a container of the powder held close up to the nose. Other suitable formulations wherein the carrier is a liquid for administration include, but are not limited to, nasal sprays, drops, or aerosols by nebulizer, an include aqueous or oily solutions of the agents.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. In some embodiments, the formulations are presented/formulated in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above-recited, or an appropriate fraction thereof, of an agent. It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, and stabilizers, etc.

In some embodiments, the compounds of the present invention are provided in unsolvated form or are in non-aqueous solutions (e.g., ethanol). The compounds may be generated to allow such formulations through the production of specific crystalline polymorphs compatible with the formulations.

In certain embodiments, the present invention provides instructions for administering said compound to a subject. In certain embodiments, the present invention provides instructions for using the compositions contained in a kit for the treatment of conditions characterized by the dysregulation of apoptotic processes in a cell or tissue (e.g., providing dosing, route of administration, decision trees for treating physicians for correlating patient-specific characteristics with therapeutic courses of action). In certain embodiments, the present invention provides instructions for using the compositions contained in the kit to treat a variety of medical conditions associated with irregular EGFR expression (e.g., HNSCC).

### C. Exemplary Administration Routes and Dosing Considerations

Various delivery systems are known and can be used to administer therapeutic agents (e.g., exemplary compounds as described in Section II above) of the present invention, e.g., encapsulation in liposomes, microparticles, microcapsules, receptor-mediated endocytosis, and the like. Methods of delivery include, but are not limited to, intra-arterial, intra-muscular, intravenous, intranasal, and oral routes. In specific embodiments, it may be desirable to administer the pharmaceutical compositions of the invention locally to the area in need of treatment; this may be achieved by, for example, and not by way of limitation, local infusion during surgery, injection, or by means of a catheter.

It is contemplated that the agents identified can be administered to subjects or individuals susceptible to or at risk of developing pathological growth of target cells and correlated conditions. When the agent is administered to a subject such as a mouse, a rat or a human patient, the agent can be added to a pharmaceutically acceptable carrier and systemically or topically administered to the subject. To determine patients that can be beneficially treated, a tissue sample is removed from the patient and the cells are assayed for sensitivity to the agent.

Therapeutic amounts are empirically determined and vary with the pathology being treated, the subject being treated and the efficacy and toxicity of the agent. When delivered to an animal, the method is useful to further confirm efficacy of the agent.

In some embodiments, in vivo administration is effected in one dose, continuously or intermittently throughout the course of treatment. Methods of determining the most effective means and dosage of administration are well known to those of skill in the art and vary with the composition used for therapy, the purpose of the therapy, the target cell being treated, and the subject being treated. Single or multiple administrations are carried out with the dose level and pattern being selected by the treating physician.

Suitable dosage formulations and methods of administering the agents are readily determined by those of skill in the art. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or non-aqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including, but not limited to, oral, rectal, nasal, topical (including, but not limited to, transdermal, aerosol, buccal and sublingual), vaginal, parental (including, but not limited to, subcutaneous, intramuscular, intravenous and intradermal) and pulmonary. It is also appreciated that the preferred route varies with the condition and age of the recipient, and the disease being treated.

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient.

Desirable blood levels of the agent may be maintained by a continuous infusion to provide a therapeutic amount of the active ingredient within disease tissue. The use of operative combinations is contemplated to provide therapeutic combinations requiring a lower total dosage of each component antiviral agent than may be required when each individual therapeutic compound or drug is used alone, thereby reducing adverse effects.

### D. Exemplary Co-Administration Routes and Dosing Considerations

The present invention also includes methods involving co-administration of the compounds described herein with one or more additional active agents. Indeed, it is a further aspect of this invention to provide methods for enhancing prior art therapies and/or pharmaceutical compositions by co-administering p300 and/or CBP-mediated transcription inhibitors (e.g., small molecules capable of binding within the KIX and/or CH1 domains of p300) (e.g., small molecules capable of binding within the KIX domain of CBP). Indeed, experiments conducted during the course of developing embodiments for the present invention determined that small molecules that target the KIX and/or CH1 domains of p300 attenuate Nanog-regulated genes and, in particular, inhibits the proliferation of cancer-initiating cells (e.g., cancer stem cells). In addition, experiments conducted during the course of developing embodiments for the present invention determined that particular isoxazolidine compounds were able to bind the KIX domain of CBP thereby attenuating binding between CBP*MLL and CBP*Jun. Accordingly, in certain other embodiments, the therapeutic methods further comprise co-administering to the subject therapeutic agents that target cancer initiating cell related pathways (e.g., therapeutic agents that target pathways involved in cancer initiating cells (e.g., Bmi-1 (see, e.g., Park, 2003; Hemmati, 2003), Notch (see, e.g., Dievart, 1999), sonic hedgehog and Wnt). In some embodiments, the therapeutic agent is salinomycin (see, e.g., Gupta, 2009). In some embodiment, the therapeutic agent is any cancer therapeutic.

In co-administration procedures, the agents may be administered concurrently or sequentially. In one embodiment, the p300 and/or CBP-mediated transcription inhibitors are administered prior to the other active agent(s). The pharmaceutical formulations and modes of administration may be any of those described above. In addition, the two or more co-administered chemical agents, biological agents or radiation may each be administered using different modes or different formulations.

The additional agents to be co-administered can be any of the well-known agents in the art for a particular disorder, including, but not limited to, those that are currently in clinical use and/or experimental use.

### VI. Other Embodiments

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The invention, now being generally described, will be more readily understood by reference to the following examples, which are included merely for purposes of illustration of certain aspects and embodiments of the present invention, and are not intended to limit the invention.

### Example 1

This example provides materials and methods information for Examples 2-4.

Plasmids

The murine-CBP expression plasmid pRC/RSV-m CBP-HA (see, e.g., Samuelson, 1997) and the 12S E1A expression plasmid, pBabe 12S E1A (see, e.g., Ptashne, 1997) were purchased from Addgene. Plasmid 0a14-MLL(2829-2883) was constructed by PCR amplification of the MLL minimal activation domain (residues 2829-2883) (see, e.g., Ernst, 2001) from a full length MLL plasmid containing an N-terminal flag tag, (F-MLL) using PCR primers 5′-GACTGGATCCCT GAAATCAGATTCAGACAATAAC-3′ (SEQ ID NO:5) and 5′-CAAGGCGGCCGCA AGACCCAATCCTTCACCAAG-3′ (SEQ ID NO:6). The PCR product was digested with BamHI and Not′ and ligated into the plasmid pCMV-Ga14 using standard molecular biology techniques. The plasmid pTKGG encoding the DNA binding domain of Ga14 fused to the minimal ligand binding domain of the glucocorticoid receptor was obtained through gift. The plasmid encoding the Jun TAD fused to the Ga14 DBD and the luciferase reporter plasmids, pGSluc and pRLSV40, were purchased from Promega. The CBP shRNA construct was prepared as previously described (see, e.g., Naidu, 2009).

Transcriptional Inhibition Assay

HeLa cells (10,000 per well) were transfected with a Firefly luciferase reporter (500 ng/mL), and Renilla (10 ng/mL) control plasmid in addition to a plasmid encoding for either the MLL (250 ng/mL) or Jun (100 ng/mL) activation domain fused to the DNA-binding domain of Ga14 in 0.1 mL Optimem. The Optimem was removed and 0.1 mL of DMEM supplemented with 10% FBS and compound was added four hours post-transfection and the luminescence was assessed after 24 h as described above.

Western Blot

HeLa cells (10,000 per well) were lysed using passive lysis buffer (Promega) with COMPLETE, EDTA-free protease inhibitors (Roche) for 10 min at room temperature on an orbital shaker. Lysates were centrifuged at 14,000 rpm for 20 min and the supernatant was mixed with 43 loading dye (Invitrogen) and BME (final concentration of 1%). The samples were then heated at 9580 for 10 min, separated by SDS-PAGE, transferred to a PVDF membrane and probed using standard conditions. Jun (sc-1694, 1:1000), cyclin D1 (sc-718, 1:1000), CBP (sc-7300, 1:500), and GAPDH (sc-47724, 1:2000) antibodies were all purchased from Santa Cruz Biotechnology.

Cell Viability Assay

MCF-7 cells (American Tissue Culture Collection) were seeded at 5000 cells per well in a 96 well plate in RPMI media supplemented with 10% fetal bovine serum and allowed to adhere (37° C. and 5% CO₂) overnight. Cells were incubated with 1b in DMSO or DMSO alone to a final DMSO concentration of 1% (v/v) for 24 hrs. WST-1 (Roche) was subsequently added to wells and the 96 well plate was incubated for 1 h at 37° C. Absorbance at 440 nM was read on a Tecan GENios Pro plate reader following 1 minute of orbital shaking. Data was corrected for wells containing media+WST-1 without cells. Each value is the average of at least three wells with the indicated error (SDOM).

¹H-¹⁵N-HSQC Binding Analysis

Uniformly labeled His₆KIX protein was expressed and purified as previously described and stored as a lyophilized powder (see, e.g., Buhrlage, 2009). A 200 μM solution of His₆KIX was prepared by dissolving the lyophilized protein, in a 9:1 H₂O:D₂O 10 mM PBS buffer containing 150 mM NaCl at pH 7.2. ¹H-¹⁵N HSQC experiments were recorded at 27° C. on an Avance Bruker 600 MHz NMR spectrometer equipped with a 5 mm cryogenic probe. An HSQC was collected in the absence of isoxazolidine 4 and compared with data recorded in the presence of 5 eq. (1 mM) of 4. Data was processed using NMRPIPE. Chemical shift assignments were based on previously reported data′ and chemical shift analyses with Sparky software were quantified (Δδ=[Δδ(¹H)²+0.1Δδ(¹⁵N)²]^{1/2}).

Small Molecule Synthesis

Unless otherwise noted, starting materials were obtained from commercial suppliers and used without further purification. CH₂Cl₂ and THF were dried by passage through activated alumina columns. All reactions were performed under a dry N₂ atmosphere unless otherwise specified. Et₃N was distilled from CaH₂. Purification by flash chromatography was carried out with E. Merck Silica Gel 60 (120-400 mesh) as previously described (see, e.g., Still, 1978). ¹H, ¹³C, and ¹⁹F NMR spectra were recording in CDC₃ at 400 MHz, 100 MHz, and 376 MHz respectively, unless otherwise specified. IR spectra were measure as thin films on NaCl plates. The preparation of compounds 1-3, 5, 51 and S6 has been previously reported (see, e.g., Minter, 2004). Note that all isoxazolidines were prepared and tested as racemic mixtures, as has been previously described (see, e.g., Minter, 2004).

### Example 2

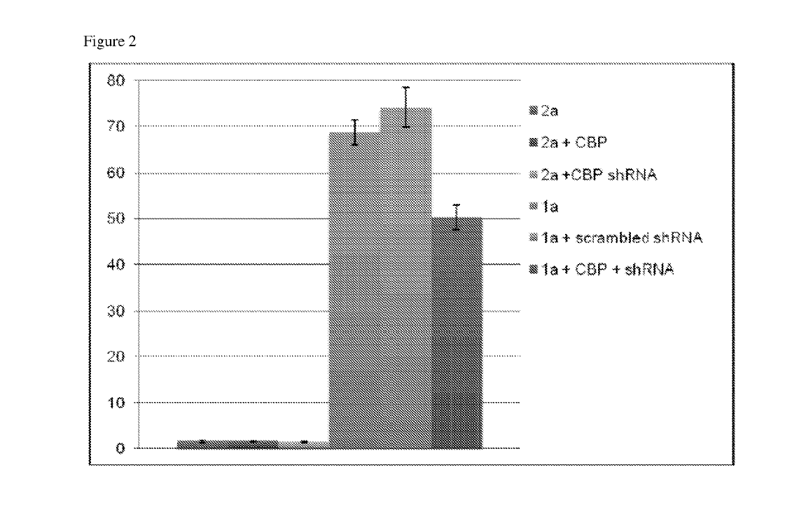

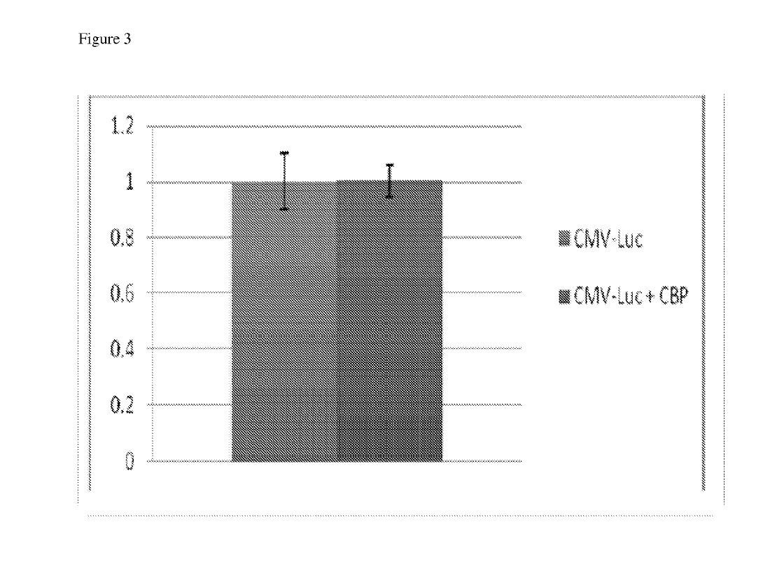

This example shows that cellular activity of isoxazolidine 1 depends upon CBP. Previously it demonstrated that amphipathic isoxazolidine 1 activates transcription and interacts with the KIX domain of CBP, while isoxazolidine 2, bearing an alkene in place of the hydroxyl functional group at C3, does not activate nor does it interact with KIX (see, e.g., Minter, 2004; Buhrlage, 2005; Rowe, 2007; Buhrlage, 2009). To assess if CBP is required for isoxazolidine 1-mediated transcriptional activity, the impact of lowering CBP concentrations shRNA knockdown was examined in the presence of 1 conjugated to the glucocorticoid receptor (GR) ligand OxDex (1a) (see, e.g., Naidu, 2009). In these experiments, cells were transfected with a luciferase reporter bearing five Ga14 binding sites and a plasmid encoding a fusion protein of the Ga14 DBD and the minimal ligand binding domain of GR in the presence of isoxazolidine 1a. In this two-hybrid assay, molecules tagged with the GR ligand OxDex will interact with the fusion protein and thus be localized to DNA (see, e.g., Liu, 2005). The knockdown of CBP with shRNA resulted in an 86 6 8% decrease (from 70-fold to 12-fold) in 1a-driven luciferase expression, while a scrambled shRNA had no effect (see FIG. 2). Additionally, the expression of exogenous CBP partially rescued the activity of isoxazolidine 1a (from 12- to 50-fold, see FIG. 2), In contrast, increased CBP concentration had no impact on the ability of isoxazolidine 2a to activate transcription, suggesting there is a direct link between CBP concentration and isoxazolidine 1a-driven activation. Additionally, a change in CBP concentration had no effect on expression of a luciferase gene driven by a CMV promoter that does not contain Ga14 binding sites (see FIG. 3). Taken together, these data reveal that, for example, the activity of isoxazolidine 1a is directly impacted by CBP concentration in the cell.

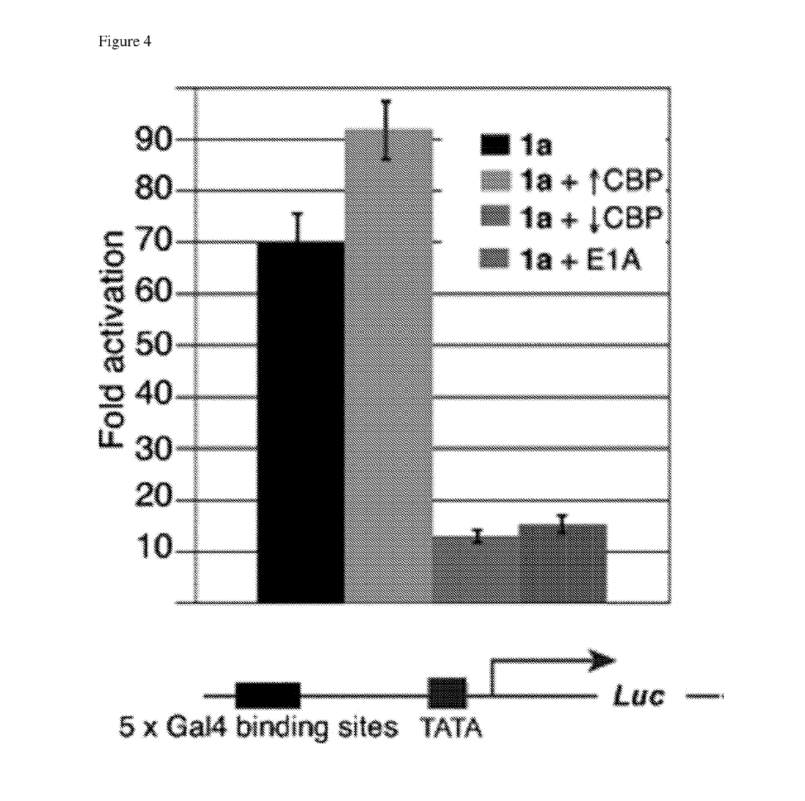

Another common strategy for altering cellular CBP availability is to use the viral protein FAA to sequester it (see, e.g., Bannister, 1995; Green, 2008; Peter, 2009; Ernst, 2001). As shown in FIG. 4, there is an 80 6 3% decrease (from 70-fold to 15-fold) in isoxazolidine 1a-mediated activation when E1A is present. In sum, modulation of transcriptional activity by affecting the availability of CBP supports the model that isoxazolidine 1a functions as a transcriptional activator recruitment of CBP in a cellular context.

### Example 3

This example shows isoxazolidine inhibition of MLL and Jun. Although a number of natural TADs interact with the KIX domain (see, e.g., Ernst, 2001; Lee, 2009; Goto, 2002; Campbell, 2002), the isoxazolidine TADs were the first examples of small molecules that interact with the site utilized by the amphipathic TADs of MLL, Jun, Tat, and Tax (see, e.g., Buhrlage, 2009). The overlapping binding footprint of isoxazolidine 1b with that of MLL and Jun should enable the small molecule to function as a competitive inhibitor of these activators. Towards this end, a series of activator competition studies were performed using proteins composed of the MLL and Jun TADs and the DBD of Ga14. Although isoxazolidine 1b is considerably smaller than the minimal TADs for MLL and Jun, it was able to inhibit the activation of both NILE and Jun in a dose-dependent manner (FIG. 5a). The EC50 for inhibition of Jun by 1b is 19 IM and for inhibition of MLL it is 361M. In addition, ESX, an activator that is not known to bind the KIX domain was not inhibited in a similar series of experiments. This is consistent with previous results performed in two ESX-driven cell lines where 1 exhibits inhibition only at high concentrations (≧50 μM) (see, e.g., Chang, 1997).

The previous reporter gene experiments demonstrated that isoxazolidine 1b inhibits the minimal TAD of Jun. It was unclear, however, if the isoxazolidine would function in a similar manner with the full-length Jun protein in an endogenous promoter context. To assess if inhibition of native Jun could be accomplished with isoxazolidine 1b, expression of the cyclin D1 protein, a key component of the cell cycle machinery, was examined (see, e.g., Fu, 2004; Arnold, 2005; Tashiro, 2007; Stacey, 2003; Albanese, 1999). Cyclin D1 expression is regulated by the AP-1 transcription factor that is a Jun homo- or a Jun-Fos heterodimer (see, e.g., Bakiri, 2000; Amanatullah, 2001; Casey, 2009; Wisdom, 1999). As illustrated in FIG. 5b, treatment of Jun-expressing MCF-7 breast cancer cells with 40 μM 1b (a concentration two-fold higher than the EC50) led to a significant decrease in cyclin D1 expression. In the same time course, expression of Jun was minimally impacted while the control, GAPDH, was unchanged. Thus, isoxazolidine 1b exerts an inhibitory effect on expression of a Jun-regulated gene not only in an artificial reporter system, but also in a native context. The demonstration of isoxazolidine 1b-mediated inhibition of a Jun-regulated gene is a key step in developing a new chemical tool for probing the Jun pathway.

### Example 4

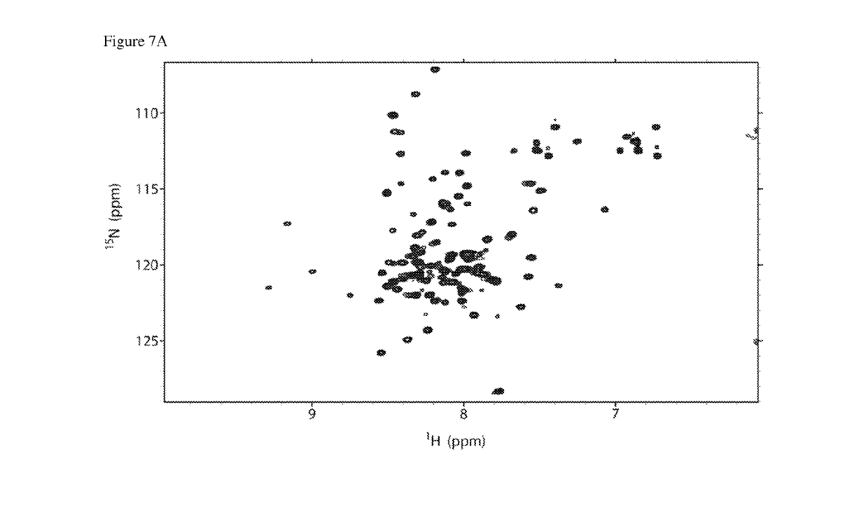

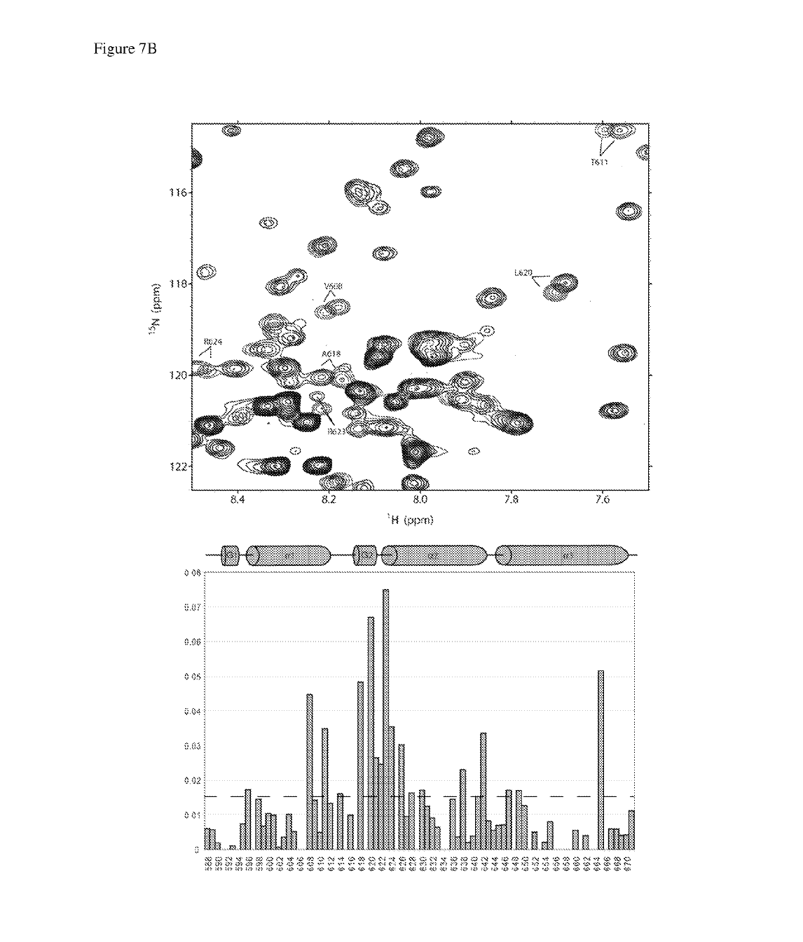

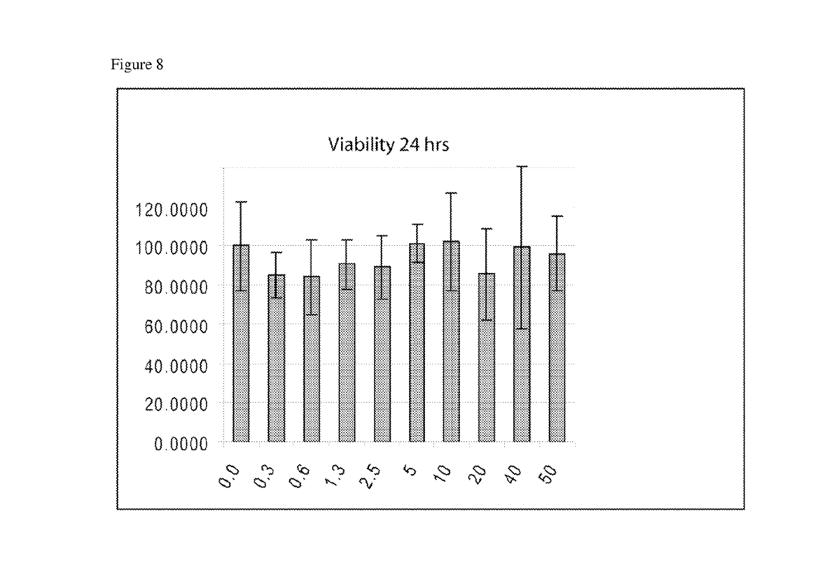

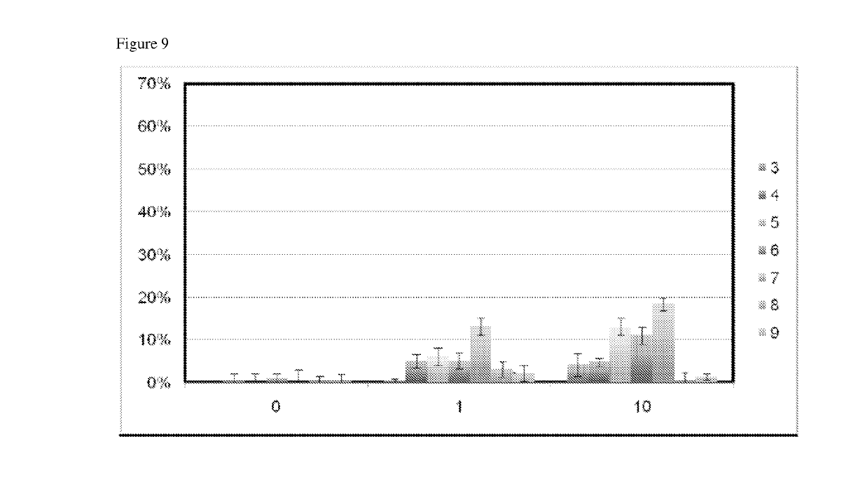

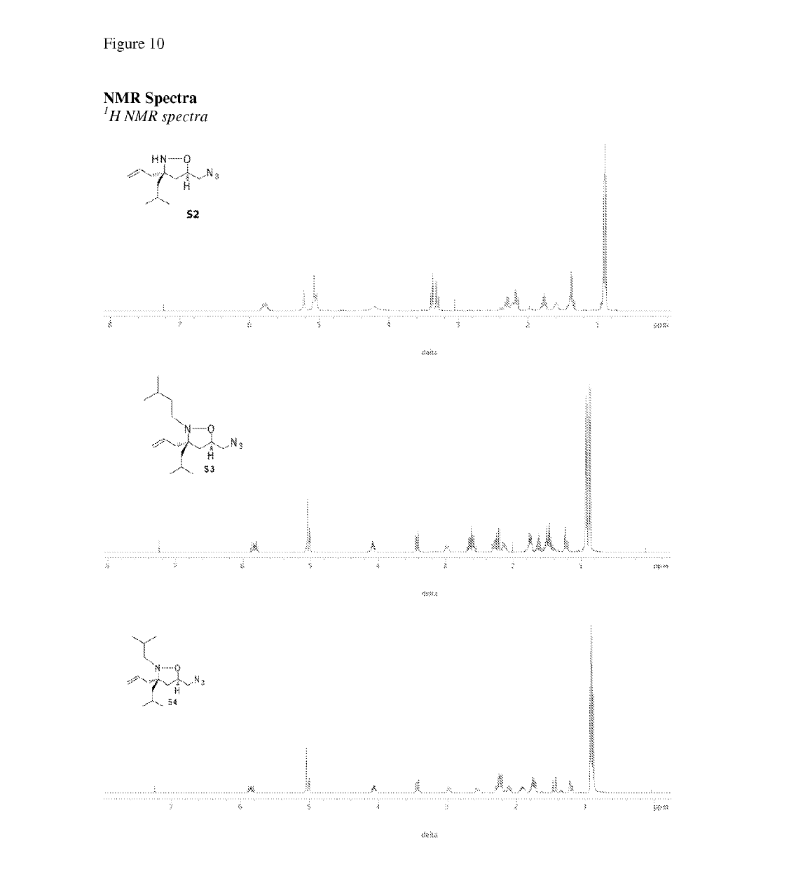

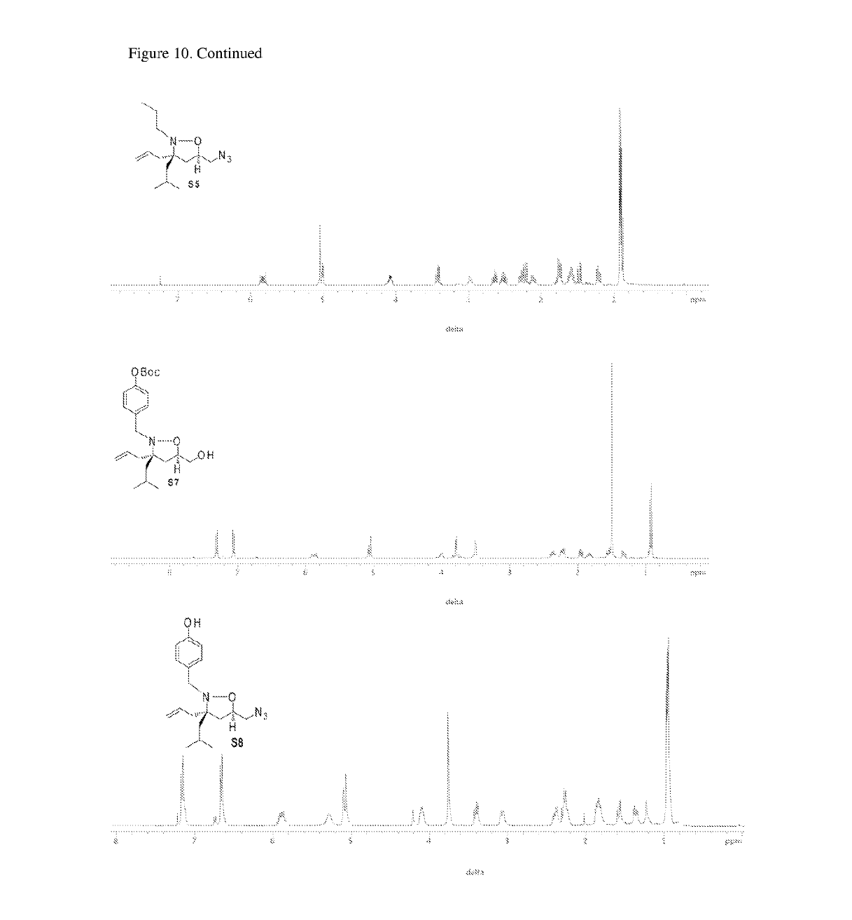

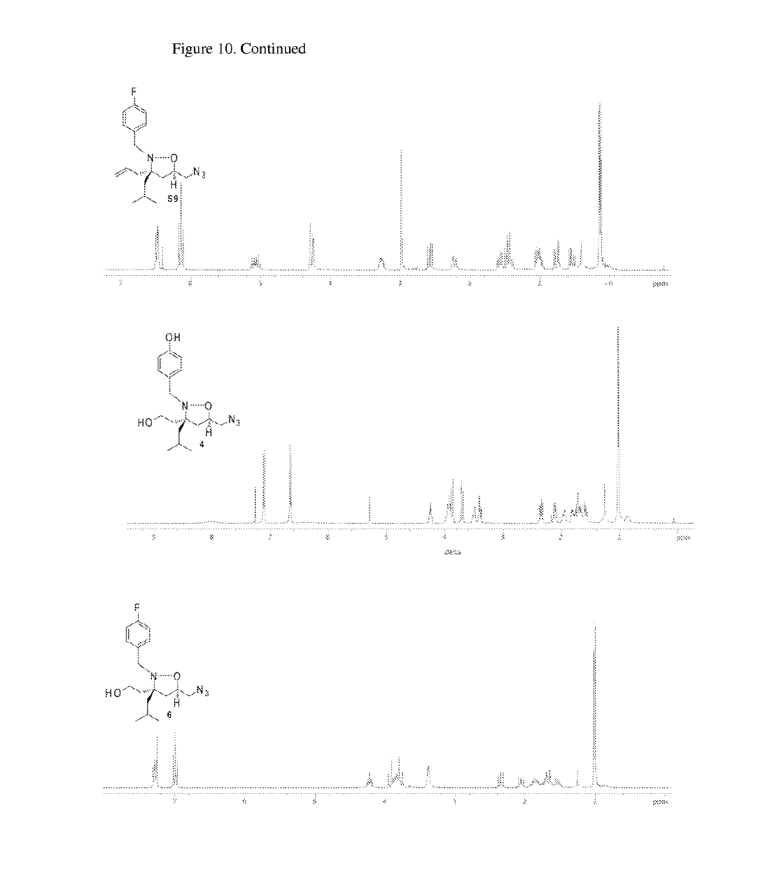

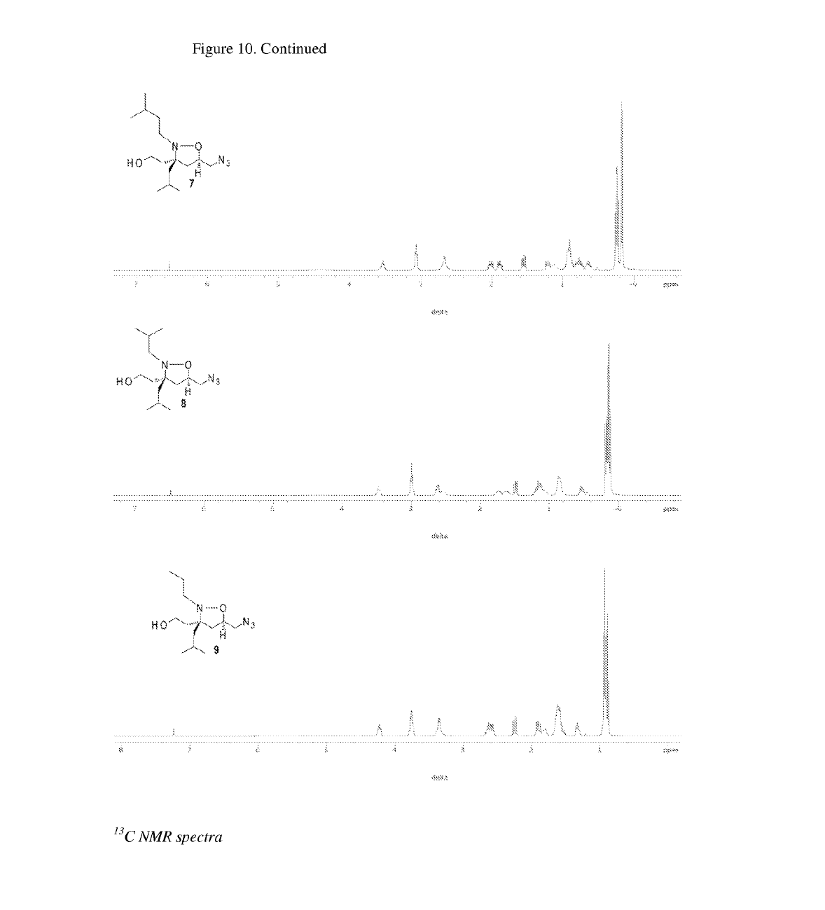

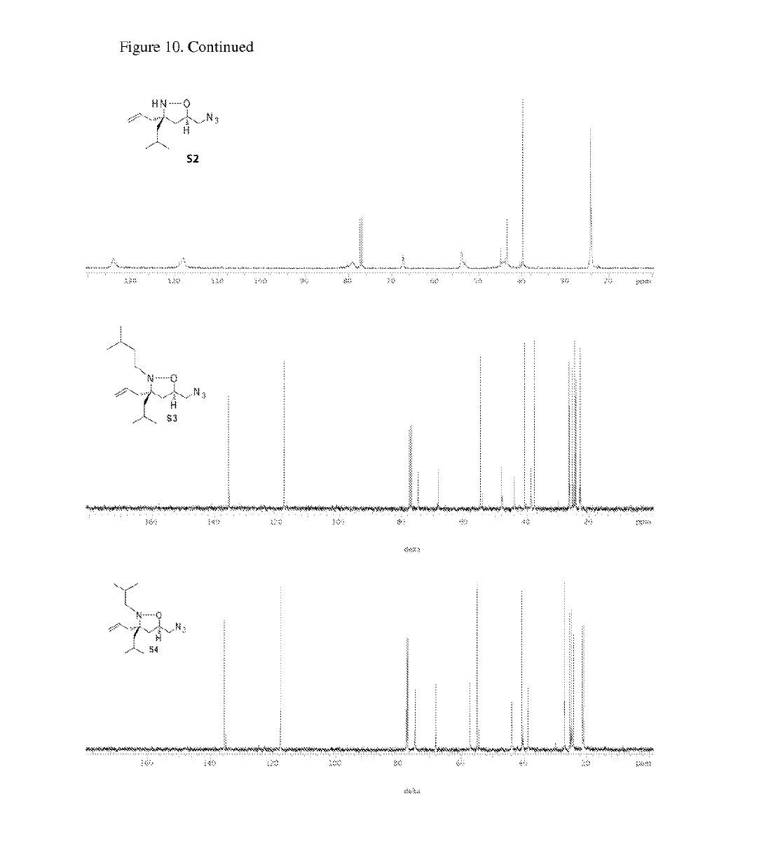

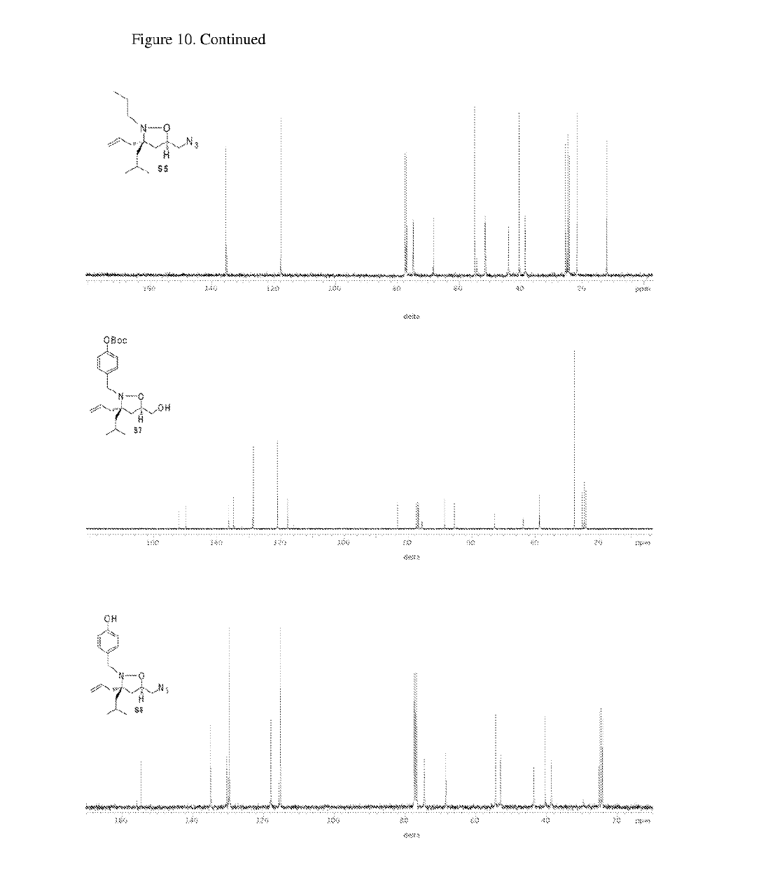

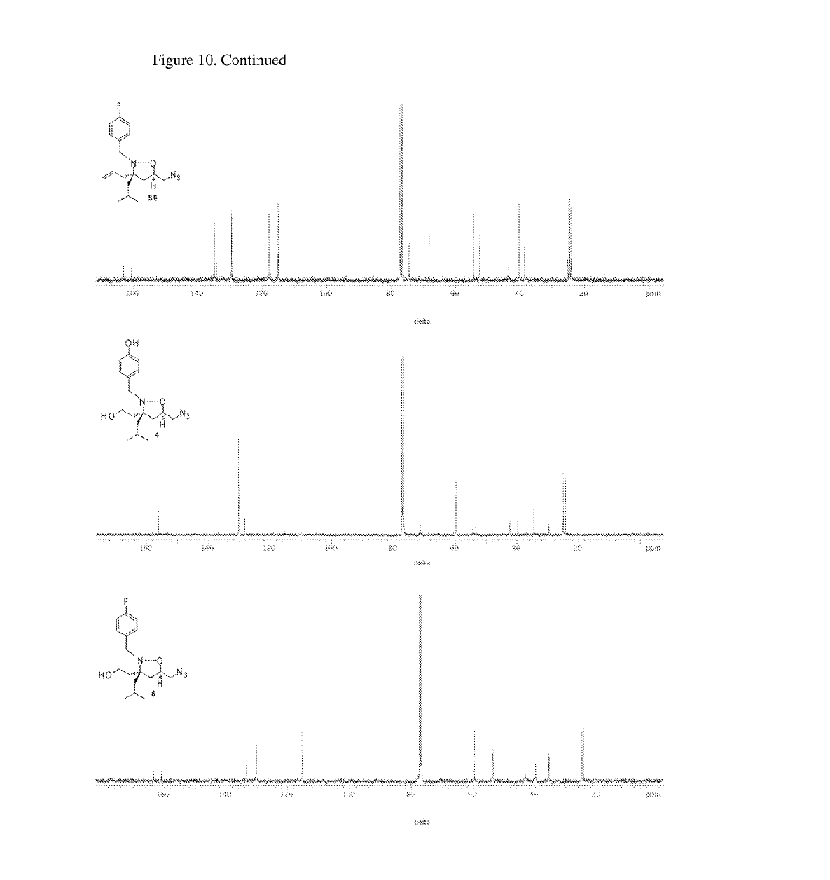

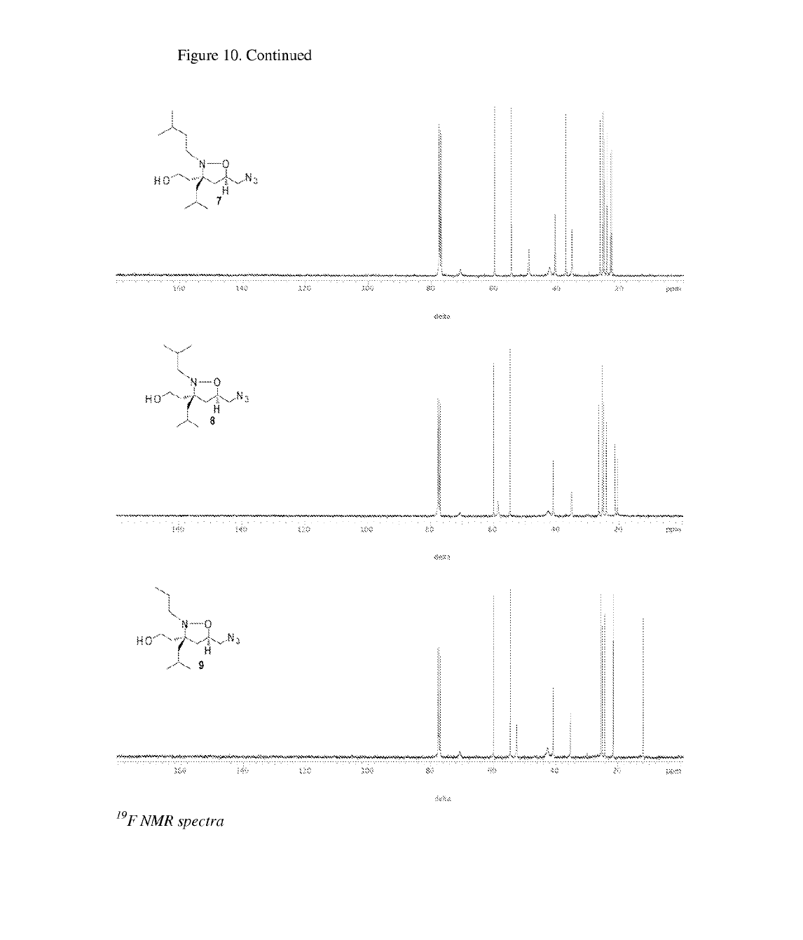

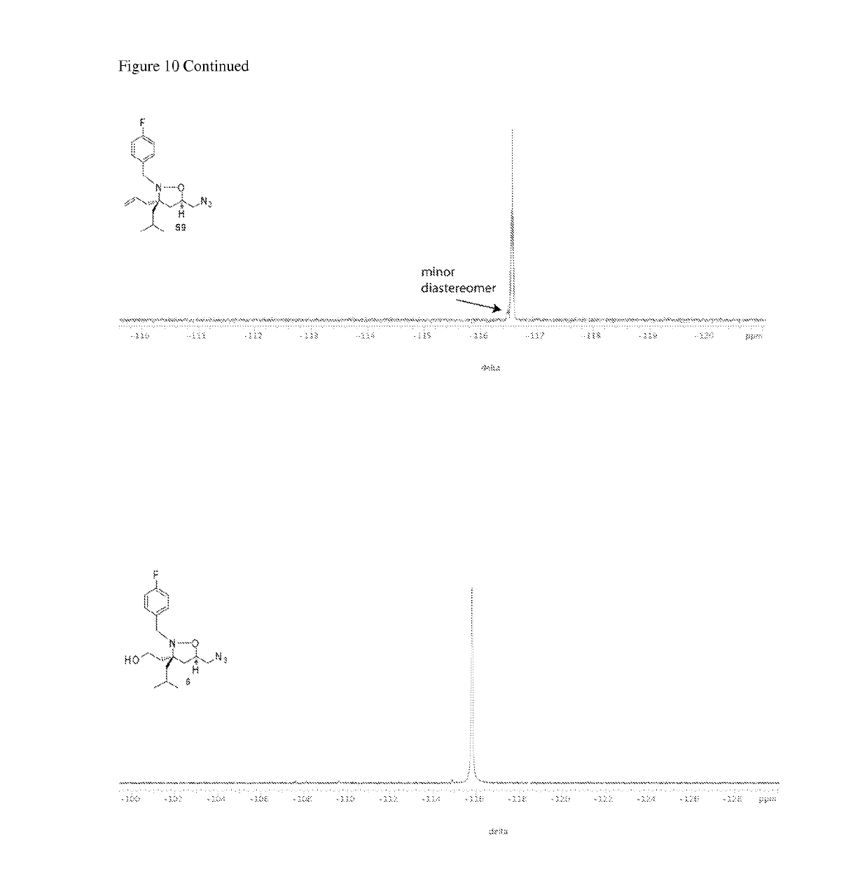

This example shows inhibition of MLL and Jun with isoxazolidine compounds. Although the results of FIG. 5a illustrate that isoxazolidine 1b serves as a transcriptional inhibitor, concentrations ≧20 μM are required. For these molecules to serve as probes of KIX function, an increase in potency is highly desirable. Like a peptide, the isoxazolidine is a modular scaffold, amenable to functionalization at the N2, C3, C4, and C5 positions. Thus, to design new molecules with increased potency, natural KIX-binding peptides for functional groups were investigated. The sequences of several natural TADs that bind the MLL/Jun/Tat/Tax site of the KLX domain (FIG. 6a) possess diverse amphipathic sequences. Whereas Tax and Jun only possess aliphatic hydrophobic side chains, Tat and MLL TADs contain aromatic side chains from tryptophan and phenylalanine, respectively. These sequence differences may contribute to the range of dissociation constants observed for these activators (MLL=2.5 μM (see, e.g., Goto, 2002), Tat=11 μM (see, e.g., Zor, 2004), Jun=30 μM (see, e.g., Amanatullah, 2001), though this has not been determined. In the case of the isoxazolidine scaffold, an isoxazolidine bearing a biphenyl substituent at the N2 position (isoxazolidine 3) does not bind the KLX domain or activate transcription (see, e.g., Cochran, 2000; Buhrlage, 2009); thus biphenyl was used as an upper limit on steric modification. It has been observed that a smaller substituent, phydroxyphenyl (isoxazolidine 4), did not negatively impact KLX binding by ¹H-¹⁵N-HSQC binding experiments (FIG. 7). Thus, to assess the functional impact of aromatic and aliphatic substituents at the N2 position of isoxazolidine 1, a small series of isoxazolidines was prepared bearing functionality similar to that of natural activators (isoxazolidines 4, 7-9; FIG. 6) and small steric (isoxazolidine 5; FIG. 6) and electronic (isoxazolidine 6; FIG. 6) modifications. While isoxazolidines 1b and 4 exhibited no measurable toxicity at concentrations ≧50 μM, the remaining analogs inhibited cell growth at concentrations above 10 μM, limiting the range of concentrations that could be examined (see FIG. 8). As expected, biphenyl 3 did not affect Jun-driven transcription; in addition, isoxazolidines 4, 8, and 9 exhibited little activity. However, as shown in FIG. 6c and FIG. 9, isoxazolidines 5, 6, and 7 inhibited Jun transcriptional activity in a dose-dependent manner. As such, these three compounds were more effective than isoxazolidine 1 (1.3- to 2-fold greater inhibition at 10 μM). The levels of inhibition achieved by these molecules is higher for Jun than for MLL, perhaps a reflection of the >10-fold difference in the dissociation constants of these two activators (FIG. 9).

### Example 5

This example shows various synthesis schemes.

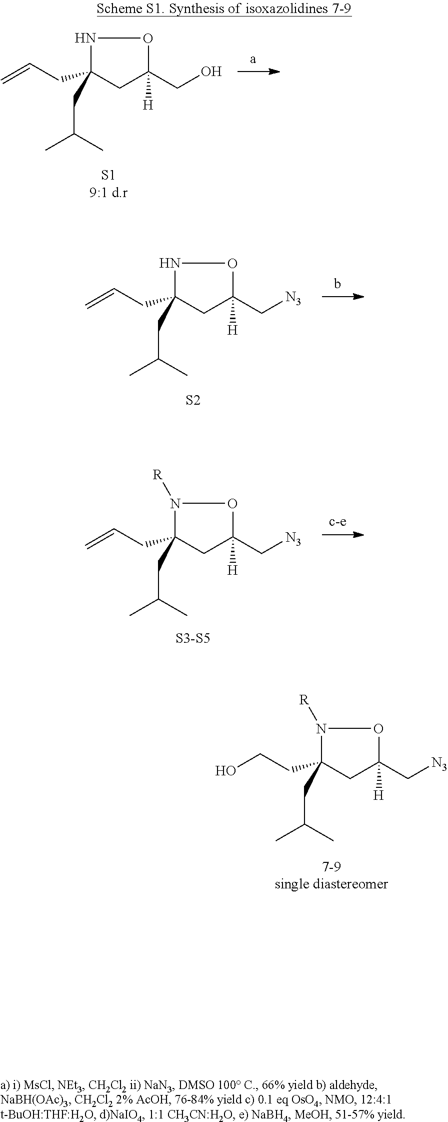

### 3-Allyl-5-(azidomethyl)-3-isobutylisoxazolidine (S2)

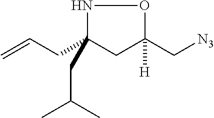

Isoxazolidine S1 (9:1 mixture of diastereomers) (see, e.g., Minter, 2004) (1.48 g, 7.42 mmol) was dissolved in CH₂Cl₂ (60 mL) and NEt₃ (1.4 mL, 10 mmol)) and cooled to 0° C. A solution of MsCl (0.70 mL, 9.0 mmol) in CH₂Cl₂ (15 mL) was then added dropwise over 10 min Once the reaction was judged complete by TLC analysis (45 min), the solution was diluted with ˜50 mL H₂O, and the aqueous phase was extracted with CH₂Cl₂ (3×). The organic phases were recombined and washed with brine (1×), dried over MgSO₄, filtered, concentrated in vacuo and the residue purified via flash column chromatography (1:1.5 hexane:ethyl acetate). The oil thus obtained was immediately dissolved in DMSO (50 mL) followed by addition of NaN₃ (4.91 g, 75.5 mmol), and heated to 100° C. Once the reaction was complete by TLC analysis (3.5 h), the reaction mixture was allowed to warm to rt. The reaction solution was diluted with ˜50 mL H₂O, and the aqueous phase was extracted with ethyl acetate (3×). The organic phases were recombined and washed with brine (1×), dried over MgSO₄, filtered, concentrated in vacuo and purified via flash column chromatography (7:1 hexane:ethyl acetate), to yield a colorless oil (1.1 g, 4.9 mmol), in 66% yield. IR: 3204, 3076 2956, 1200, 1639, 1438, 1366, 1277, 1157, 1041 cm⁻¹, ¹H NMR of major diastereomer (400 MHz, CDC₃): δ 0.89 (d, 3H, J=6.9); 0.91 (d, 3H, J=6.7); 1.37-1.44 (m, 2H); 1.40-1.80 (m, 1H); 1.77 (m, 1H, J=6.7); 2.13-2.25 (m, 1H), 2.16 (dd, 1H, J=14.2, 7.8); 2.31 (dd, 1H, J=14.2, 6.6); 3.30 (dd 1H, J=12.9, 5.3); 3.39 (dd, 1H, J=12.9, 3.6); 4.21 (br s, 1H); 5.04-5.08 (m 2H); 5.78 (m, 1H) ¹³C NMR of major diastereomer (100 MHz, CDC₃): δ 134.26, 118.04, 67.48, 67.30, 53.93, 44.94, 43.95, 43.49, 39.86, 24.29, 24.22 HRMS: (ESI) calc for [C₁₁H₂₀N₄O+Na]⁺ 247.1535. found: 247.1540

### 3-Allyl-5-(azidomethyl)-3-isobutyl-2-isopentylisoxazolidine (S3)

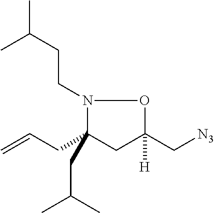

Isoxazolidine S2 (85.6 mg, 0382 mmol) was dissolved in 4 mL of a solution of CH₂Cl₂and 2% AcOH (v/v) followed by addition of isovaleraldehyde (0.41 mL, 3.8 mmol) and Na(OAc)₃BH (410 mg, 1.9 mmol) and left to stir at room temperature for 3.5 h. The reaction mixture was diluted with H₂O and the aqueous phase was extracted with CH₂Cl₂ (3×). The organic phases were recombined, washed with brine (1×), dried over MgSO₄, filtered, concentrated in vacuo and the residue was purified via flash column chromatography (25:1 hexane:ethyl acetate) to yield a yellow oil (88 mg, 0.30 mmol) in 79% yield of S3. IR: 2955, 2929, 2869, 2098, 1637, 1467, 1366, 1279, 1168 cm^{-1 1}H NMR of major diastereomer (400 MHz, CDC₃): δ 0.87 (d, 3H, J=6.6); 0.87 (d, 3H, J=6.5); 0.91 (d, 3H, J=6.7); 0.92 (d, 3H, J=6.6); 1.22 (m, 1H); 1.48-1.53 (m, 3H); 1.63 (in, 1H); 1.74 (m, 2H); 2.13 (m, 1H); 2.22 (dd, 1H, J=12.5 8.4); 2.29 (d, 1H, J=13.9, 7.3); 2.59-2.66, (m, 1H); 3.00 (m 1H); 3.43 (dd, 1H, J=12.7, 4.9); 4.08, (in, 1H); 5.01-5.05 (m, 2H); 5.83 (m, 1H) ¹³C NMR of major diastereomer (100 MHz, CDC₃): 135.46, 117.68, 74.94, 68.32, 54.83, 47.94, 43.84, 40.6:2, 38.60. 37.47, 26.35, 25.45, 24.59, 24.28. 22.90. 22.79 HRMS: (ESI) calc for [C₁₆H₃₀N₄₀+Na]⁺ 317.2317. found: 317.2305

### 3-Allyl-5-(azidomethyl)-2,3-diisobutylisoxazolidine (S4)

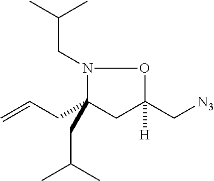

S2 (85.6 mg, 0382 mmol) was subjected to the same reductive amination conditions described for S3 resulting in 76% yield of S4 as a colorless oil (82 mg, 0.29 mmol). IR: 3072, 2956, 2927, 2870, 2099 1637, 1466 1386, 1367, 1284 cm^{-1 1}H NMR of major diastereomer (400 MHz, CDC₃): δ 0.88-0.93 (in, 1211); 1.21 (dd, 1H, J=14.5, 5.9); 1.45 (dd, 1H, J=14.4, 5.3); 1.72-1.79 (m, 2H); 1.91 (m, 1H); 2.10 (dd, 1H, J=13.9, 7.7); 2.20-2.30 (m, 3H); 2.97 (dd, 1H, J=12.8, 8.1); 2.97 (d 1H, J=10); 3.44 (dd, 1H, J=12.7, 8.0); 4.07 (m, 1H); 5.02 (m, 2H); 5.84 (m, 1H) ¹³C NMR of major diastereomer (100 MHz, CDC₃): δ 133.58, 117.61, 74.63, 68.04, 57.19, 54.92, 43.89, 40.55, 38.59, 27.07, 25.42, 24.77, 24.19, 21.34, 20.90 HRMS: (ESI) calc for [C₁₅H₂₈N₄O+Na]⁺ 303.2161. found: 303.2160

### 3-Allyl-5-(azidomethyl)-3-isobutyl-2-propylisoxazolidine, (S5)

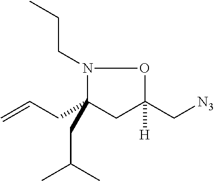

S2 (85.6 mg, 0382 mmol) was subjected to the same reductive amination conditions described for S3 resulting in 84% yield of a colorless oil (86 mg, 0.32 mmol) IR: 3076, 2959, 2930, 2872, 2099, 1638, 1454, 1360, 1277, 1168, 1058 cm^{-1 1}H NMR of major diastereomer (400 MHz, CDC₃): δ 0.88-0.94 (m, 9H); 1.21 (dd, 1H, J=14.5, 6.6); 1.48 (dd, 1H, J=14.4, 4.7); 1.77 (m 2H); 1.73-1.80 (m 2H); 2.12 (dd, 1H, J=13.7, 7.3); 2.10-131 (m, 2H); 2.50 (m, 1H); 2.64 (m, 1H); 2.98 (d, 1H J=12.5); 3.43 (m, 1H); 5.00-5.04 (m, 2H); 5.83 (m, 1H) ¹³C NMR of major diastereomer (100 MHz, CDC₃): δ 135.47, 117.68, 74.75, 68.19, 54.84, 51.48, 43.81, 40.55, 38.60, 25.44, 24.63, 24.23, 21.76, 12.11 HRMS: (ESI) calc for [C₁₄H₂₆N₄O+Na]⁺ 289.2004. found: 289.2005

### General Procedure for Double Bond Oxidative Cleavage

### 5-(Azidomethyl)-3-isobutyl-2-isopentylisoxazolidin-3-yl)ethanol (7)

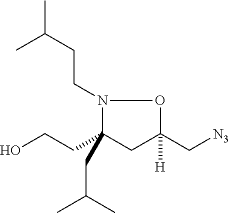

Isoxazolidine S3 (80.3 g, 0.273 mmol) was dissolved in a 12:4:1 mixture of t-BuOH:THF:H₂O (4 mL) followed by addition of 270 μL of a 2.5% solution of OsO₄ (0.027 mmol) and NMO (49.4 mg, 0.422 mmol) and left to stir for 6 h. The reaction was quenched with Na₂SO₄ and left to stir for 1.5 h. The reaction mixture was diluted with H₂O and the aqueous phase was extracted with ethyl acetate (3×). The organic phases were recombined, washed with brine (1×), dried over MgSO₄, filtered and concentrated in vacuo. The resulting oil was redissolved in a 1:1 solution of CH₃CN (5 mL) followed by addition of NaIO₄ (77 mg, 0.33 mmol) and left to stir for 1.5 h upon consumption of starting material as judged by TLC. The reaction mixture was diluted with H₂O and the aqueous phase was extracted with ethyl acetate (3×), washed with brine (1×), dried over MgSO₄, filtered and concentrated in vacuo to yield a colored oil. The oil was redissolved in methanol (5 mL) and the aldehyde was subsequently reduced with NaBH₄ (17.1 mg, 2.04 mmol). The reaction was complete after 1 h as indicated by TLC analysis. The reaction was cooled to 0° C. and quenched with water. The reaction mixture was further diluted with H₂O and the aqueous phase was extracted with ethyl acetate (3×). The organic phases were recombined and washed with brine (1×), dried over MgSO₄, concentrated in vacuo and the residue purified via flash column chromatography (3:1 hexane:ethyl acetate) to yield a single diastereomer of 7 as a colorless oil (0.042 g, 0.14 mmol) in 51% yield over three steps. IR: 3368.2 (b) 2956, 2870, 2100, 1468, 1367, 1280, 1168, 1048 cm^{-1 1}H NMR (400 MHz, CDC₃): δ 0.087 (d, 6H, J=6.4); 0.93 (d, 3H, J=7.4); 0.95 (d, 3H, J=6.8); 0.93 (d, 3H, J=7.4); 1.33 (m, 1H); 1.46 (m, 2H); 1.46 (m, 2H); 1.52-1.62 (m, 4H); 1.81 (br m, 1H); 1.91 (dd, 1H, J=12.7, 8.5); 2.25 (dd, 1H, J=12.6, 7.9); 2.59 (m, 1H); 2.72 (m, 1H); 3.48 (m, 2H); 3.76 (t, 2H, J=5.3); 4.23 (m, 1H) ¹³C NMR (100 MHz, CDC₃): δ 70.85, 59.91, 54.55, 49.03, 42.40, 40.69, 37.16, 35.25, 26.34, 25.45, 25.04, 24.19, 23.03, 22.61 HRMS: (ESI) calc for [C₁₅H₃₀N₄O₂+H]⁺ 299.2447. found: 289.2442

### 5-(Azidomethyl)-2,3-diisobutylisoxazolidin-3-yl)ethanol, (8)

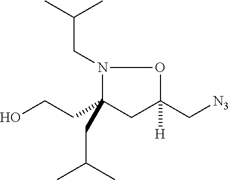 [SMILES: CC(C)CN1O[C@@H](CN=[N+]=[N-])C[C@@]1(CCO)CC(C)C]

Isoxazolidine S4 (81.5 mg, 0.291 mmol) was subjected to the general procedure for double bond oxidative cleavage described above to yield a single diastereomer of 8 as a colorless oil (47 mg, 0.16 mmol) in 57% yield over the three steps. IR: 3380 (b), 2957, 2927, 2871, 2101, 1467, 1387, 1368, 1283, 1169, 1128, 1048 cm⁻¹. ¹H NMR (400 MHz, CDC₃): δ 0.88 (d, 6H, J=7.0); 0.92 (d 3H, J=6.0); 0.94 (d 3H, J=6.5); 1.29 (dd, 1H, J=14.1, 7.9); 1.61 (m, 3H); 1.82-1.92 (m, 3H); 2.25 (dd, 1H, J=12.5, 7.8); 2.37 (br m, 1H); 2.49 (br m, 1H); 3.31 (br m, 1H); 3.39 (dd, 1H, J=12.4, 6.2); 3.74 (t 2H, J=5.5); 4.23 (m, 1H) ¹³C NMR (100 MHz, CDC₃): δ 70.56, 59.90, 58.56, 54.75, 42.43, 40.94, 35.09, 26.49, 25.44, 25.02, 24.07, 21.31, 20.60 ESI calc for [C₁₄H₂₈N₄O₂+H]⁺ 285.2291. found: 289.2299

### 5-(Azidomethyl)-3-isobutyl-2-propylisoxazolidin-3-yl)ethanol (9)

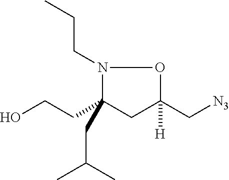 [SMILES: CCCN1O[C@@H](CN=[N+]=[N-])C[C@@]1(CCO)CC(C)C]

Isoxazolidine S5 (85.6 mg, 0.322 mmol) was subjected to the general procedure for oxidative cleavage of a double bond described above to yield a single diastereomer of 9 (49 mg, 0.18 mmol) as a colorless oil in 57% yield over three steps IR: 3382 (b), 2958, 2932, 2872, 2099, 1457, 1382, 1367, 1278, 1167, 1047 cm^{-1 1}H NMR (400 MHz, CDC₃): 0.87-0.94 (m 9H); 1.32 (dd, 1H, J=14.1, 7.6); 1.58-1.62 (m, 5H); 1.64 (br m, 1H); 1.90 (dd, 1H, J=12.6, 8.4); 2.25 (dd, 1H, J=12.5, 7.9); 2.61 (m, 2H) 3.35 (m, 2H); 3.75 (m, 2H); 4.22 (m, 1H) ¹³C NMR (100 MHz, CDC₃): δ 77.43, 70.69, 59.87, 54.49, 52.43, 42.47, 40.64, 35.21, 25.40, 24.97, 24.17, 21.43, 11.88 ESI calc for [C₁₃H₂₆N₄O₂+H]⁺ 271.2134. found: 271.2128

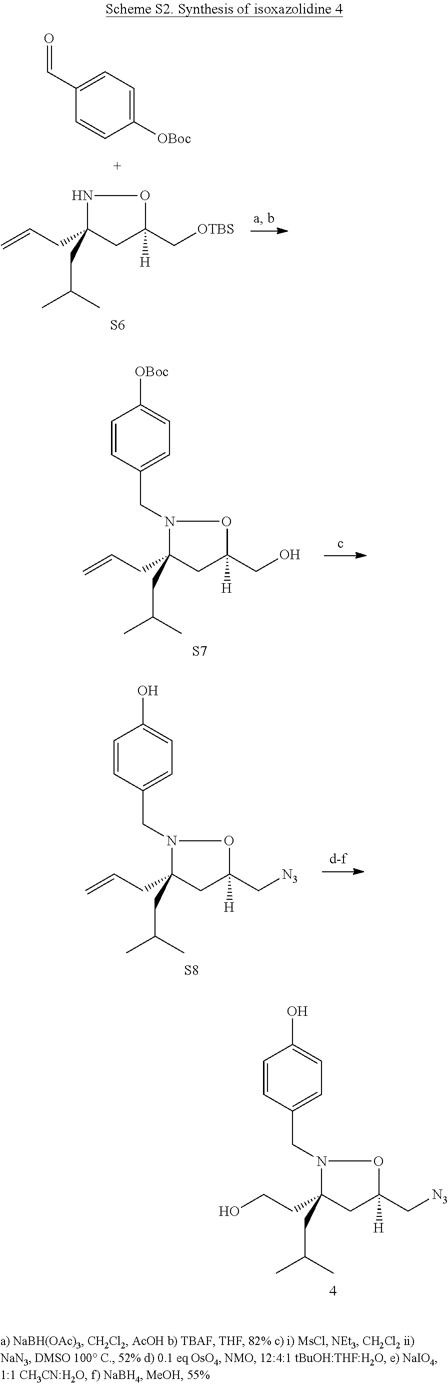

### 3-Allyl-5-(hydroxymethyl)-3-isobutylisoxazolidin-2-yl)methyl)phenyl tert-butyl carbonate (S7)

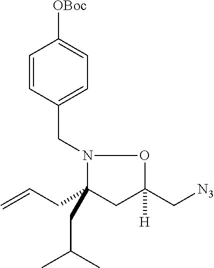

Isoxazolidine S6 (0.1597 g, 0.5090 mmol) was dissolved in 8 mL of a solution of CH₂Cl₂ followed by addition of tert-butyl (4-formylphenyl) carbonate (0.2345 g, 1.056 mmol) and Na(AcO)₃BH (0.41 g, 1.9 mmol) to yield a cloudy solution. Five drops of AcOH were added, resulting in a clear solution. The reaction was left to stir at room temperature overnight. An additional two equivalents of tert-butyl (4-formylphenyl) carbonate were then added. The reaction mixture was diluted with ˜10 mL of H₂O and the aqueous phase was extracted with CH₂Cl₂ (3×). The organic phases were recombined and washed with brine (1×), dried over MgSO₄, filtered and concentrated in vacuo. The crude oil was redissolved in THF (10 mL) followed by addition of 3.1 mL of 1M TBAF (3.1 mmol) and left to stir for 1 h. The reaction mixture was diluted with H₂O and the aqueous phase was extracted with ethyl acetate (3×). The organic phases were recombined and washed with brine (1×), dried over MgSO₄, filtered and concentrated in vacuo. The crude product was purified via flash column chromatography (3:1 hexane:ethyl acetate) yielding a single diastereomer of S7 as a colorless oil (0.17 g, 0.42 mmol) in 82% yield. ¹H NMR (400 MHz, CDC₃): δ 0.91 (d, 3H, J=7.5); 0.93 (d, 3H, J=6.8); 1.32 (dd, 1H, J=14.4, 6.7); 1.50 (s, 9H); 1.55 (dd, 1H, J=14.4, 4.6); 1.83 (m 1H); 1.95 (dd, 1H, J=12.5, 6.2); 2.19-2.25 (m, 2H); 2.32 (br s, 1H); 2.38 (dd, 1H, J=13.9, 7.3); 3.51 (m, 2H); 3.77 (d, 1H, J=14.4); 3.82 (d, 1H, J=14.4); 4.00 (m, 1H); 5.04-5.08 (m, 2H); 5.88 (m, 1H) 7.06 (d, 2H, J=8.4); 7.31 (d, 2H, J=8.4) ¹³C NMR (100 MHz, CDC₃): δ 152.09, 150.05, 136.55, 135.13, 128.89, 121.20, 117.91, 83.46, 68.59, 65.52, 52.96, 43.92, 38.91, 38.78, 27.81, 25.37, 24.65, 24.20 ESI calc for [C₂₃H₃₅NO₅+Na]⁺ 428.2413. found: 428.2409

### 3-Allyl-5-(azidomethyl)-3-isobutylisoxazolidin-2-yl)methyl)phenol (S8)

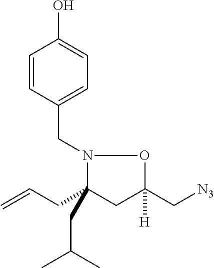 [SMILES: C=CC[C@@]1(CC(C)C)C[C@H](CN=[N+]=[N-])ON1Cc1ccc(O)cc1]

Isoxazolidine S7 (0.1696 g, 0.4180 mmol) was dissolved in CH₂Cl₂ (10 mL) and NEt₃ (0.23 mL, 1.7 mmol)) followed by addition of MsCl (0.13 mL, 1.7 mmol). Once the reaction was judged complete by TLC analysis (80 min), the reaction solution was diluted with ˜10 mL H₂O, and the aqueous phase was extracted with CH₂Cl₂ (3×). The organic phases were recombined and washed with brine (1×), dried over MgSO₄, filtered and concentrated in vacuo. The crude oil was immediately taken up in DMSO (4 mL) followed by addition of NaN₃ (0.2816 g, 4.332 mmol), and heated to 100° C. and left to stir for 20 h. Once the reaction was complete by TLC analysis, the reaction was cooled to room temperature. The reaction solution was diluted with H₂O, and the aqueous phase was extracted with CH₂Cl₂ (3×). The organic phases were recombined and washed with brine (1×), dried over MgSO₄, filtered, concentrated in vacuo and the residue purified via flash column chromatography (4:1 hexane:ethyl acetate), to yield S8 as a colorless oil (0.071 g, 0.22 mmol) in 52% yield. ¹H NMR (400 MHz, CDC₃): δ 0.94-0.97 (m 6H); 1.37 (dd, 1H, J=14.4, 6.4); 1.58 (dd, 1H J=14.4, 5.0); 1.84 (m, 2H); 2.03 (m, 2H); 2.39 (dd, 1H J=13.9, 7.1); 3.07 (m, 1H); 3.40 (dd, 1H, J=12.7, 7.2); 3.76 (br s, 2H); 4.10 (m, 1H) 5.07-5.28 (m, 2H); 5.28 (br s, 1H); 5.90 (br s, 1H); 6.66 (d, 2H, J=8.4); 7.16 (d, 2H, J=8.5) ¹³C NMR (100 MHz, CDC₃): δ 154.79, 135.09, 130.69, 129.77, 118.13, 115.44, 74.78, 68.67, 54.62, 53.24, 43.75, 40.66, 38.90, 25.41, 24.82, 24.40 ESI calc for [C₁₈H₂₆N₄O₂+Na]⁺ 353.1953. found: 353.1958

### 5-(Azidomethyl)-3-(2-hydroxyethyl)-3-isobutylisoxazolidin-2-yl)methyl)phenol (4)

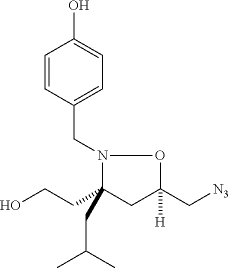 [SMILES: CC(C)C[C@]1(CCO)C[C@H](CN=[N+]=[N-])ON1Cc1ccc(O)cc1]

Isoxazolidine S8 (0.0527 g, 0.160 mmol) was subjected to the general procedure for an oxidative C3 olefin to primary alcohol transformation described above to yield 4 as a white solid (0.0295 g, 0.088 mmol) in 55% yield over 3 steps. ¹H NMR (400 MHz, CDC₃): δ 1.02 (d, 6H, J=6.3); 1.58 (dd, 1H, J=12.9, 5.9); 1.68-1.82 (m, 5H); 1.96 (m, 1H); 2.11 (dd, 1H, J=12.5, 8.6); 2.35 (dd, 1H, J=12.9, 7.9); 2.11 (dd, 1H, J=12.5, 8.6); 3.39 (dd, 1H, J=12.9, 4.7); 3.50 (dd, 1H, J=12.9, 3.5); 3.70 (d, 1H, J=13.3); 3.88 (d, 1H, J=13.3); 3.91-3.98 (m, 2H); 4.25 (m, 1H, J=4.1); 6.66 (d, 2H, J=8.2); 7.11 (d, 2H, J=8.2); 8.00 (br s, 1H) ¹³C NMR (100 MHz, CDC₃): δ 156.10, 130.15, 128.17, 115.50, 71.37, 59.72, 54.19, 53.33, 42.41, 39.69, 34.49, 29.69, 25.22, 24.96, 24.27 ESI calc for [C₁₇H₂₆N₄O₃+Na]⁺ 357.1903. found: 357.1899 X-ray quality crystals of 4 were obtained via slow evaporation of a CH₂Cl₂ from a solution of hexane and CH₂Cl₂. The x-ray diffraction structure is shown below:

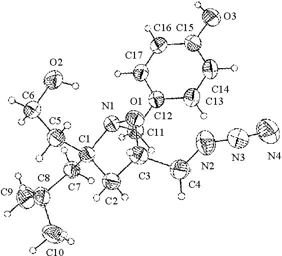

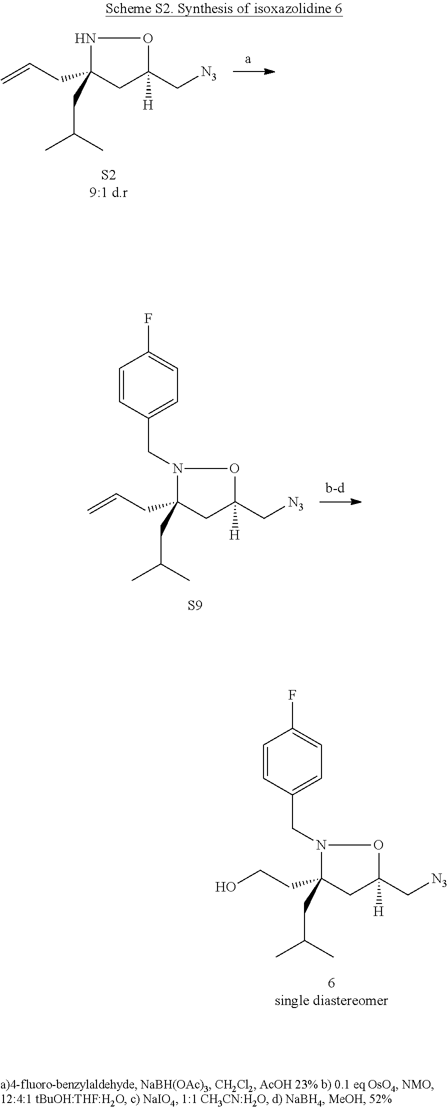

### 3-Allyl-5-(Azidomethyl)-2-(4-fluorobenzyl)-3-isobutylisoxazolidine (S9)

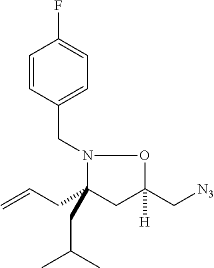 [SMILES: C=CC[C@@]1(CC(C)C)C[C@H](CN=[N+]=[N-])ON1Cc1ccc(F)cc1]

Isoxazolidine S2 (0.103 g, 0.460 mmol) was dissolved in 5 mL of a solution of CH₂Cl₂ followed by addition of 4-fluorobenzaldehyde ((0.24 mL, 2.3 mmol) and Na(OAc)₃BH (0.472 g, 2.24 mmol) and 5 drops of AcOH and left to stir at room temperature for 24 h. The reaction mixture was diluted with H₂O and the aqueous phase was extracted with CH₂Cl₂ (3×). The organic phases were recombined, washed with brine (1×), dried over MgSO₄, filtered, concentrated in vacuo and the residue was purified via flash column chromatography (20:1 hexane:ethyl acetate) to yield S9 as a colorless oil (35 mg, 0.10 mmol) in 23% yield. ¹H NMR (400 MHz, CDC₃) of major diastereomer: δ 0.97 (d, 3H, J=6.4); 0.98 (d, 3H, J=6.7); 1.36 (dd, 1H, J=14.6, 6.4); 1.60 (dd, 1H, J=14.6, 5.0); 1.63-1.90 (m, 2H); 2.22-2.31 (m, 2H); 2.37 (dd, 1H, J=18.2, 7.3); 3.06 (dd, 1H, J=12.9, 3.8); 3.41 (dd, 1H, J=12.9, 3.8); 3.82 (s, 2H); 4.10 (m, 1H); 5.08-5.12 (m, 2H); 5.90 (m, 1H); 6.98 (t, 2H, 8.8); 7.32 (dd, 2H, J=8.7, 5.5) ¹³C NMR (100 MHz, CDC₃) of major diastereomer 161.87 (d, J=244), 134.84, 134.32, 129.58 (d, J=7.8), 117.94, 114.95, (d, J=21), 74.55, 68.32, 54.38, 52.65, 43.58, 40.24, 38.69, 25.22, 24.61, 24.15 ¹⁹F NMR (376 MHz, CDC₃): of major diastereomer 8 ESI calc for [C₁₈H₂₅FN4O+H]⁺ 333.2091. found: 333.2084

### 5-(Azidomethyl)-2-(4-fluorobenzyl)-3-isobutylisoxazolidin-3-yl)ethanol (6)

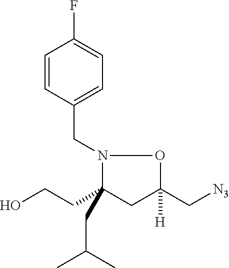 [SMILES: CC(C)C[C@]1(CCO)C[C@H](CN=[N+]=[N-])ON1Cc1ccc(F)cc1]

Isoxazolidine S9 (70.8 mg, 0.213 mmol) was subjected to the general procedure for double bond oxidative cleavage described above to yield a single diastereomer of 6 as a colorless oil (42 mg, 0.14 mmol) in 52% yield. ¹H NMR (400 MHz, CDC₃): δ 0.94 (d, 6H, J=6.4); 1.51 (dd, 2H, J=13.5, 6.5); 1.79-2.07 (m, 4H); 2.04 (dd, 1H, J=12.6 7.9); 2.33 (dd, 1H, J=12.9, 7.9); 3.37 (d, 2H, J=4.7); 3.76 (d, 1H, J=14.0); 3.80 (m, 2H); 3.91, (d, 1H, J=14.0); 4.20 (m, 1H); 6.98 (t, 2H; J=8.8); 7.27 (dd, 2H, J=8.5, 5.6) ¹³C NMR (100 MHz, CDC₃) 162.05 (d, J=245), 133.39, 130.18 (d, J=8.2), 115.20, (d, J=21.1), 70.44, 59.63, 53.56, 43.09. 39.93, 35.61, 25.22, 24.86, 24.33 ¹⁹F NMR (376 MHz, CDC₃): δ 115.81 ESI calc for [C₁₇H₂₅FN₄O₂+Na]⁺ 359.1859. found: 359.1862

### Example 6

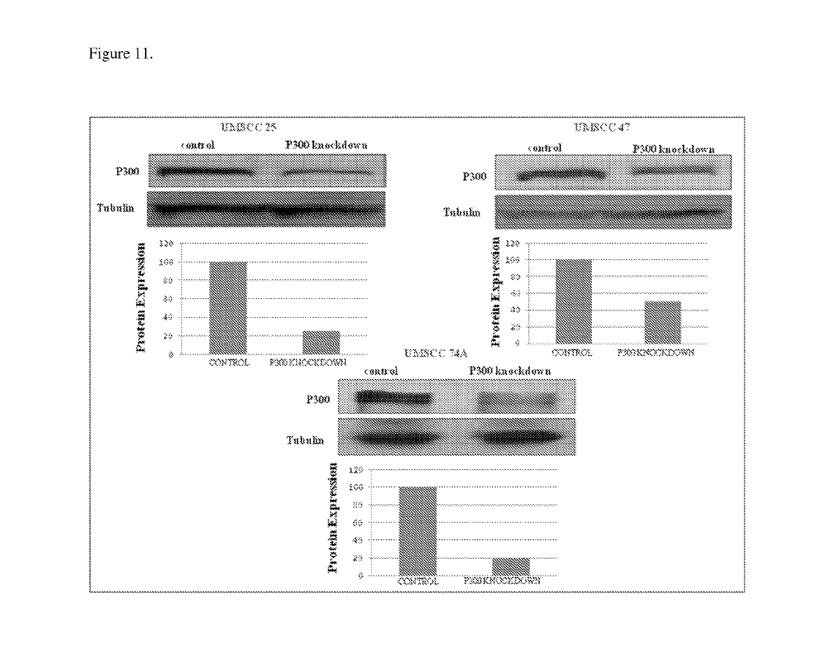

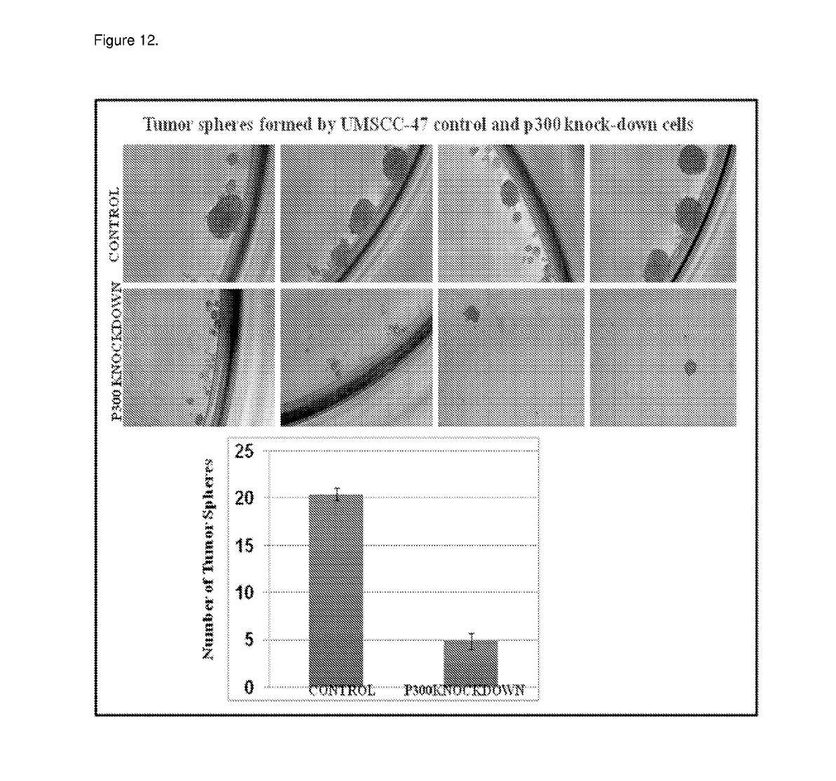

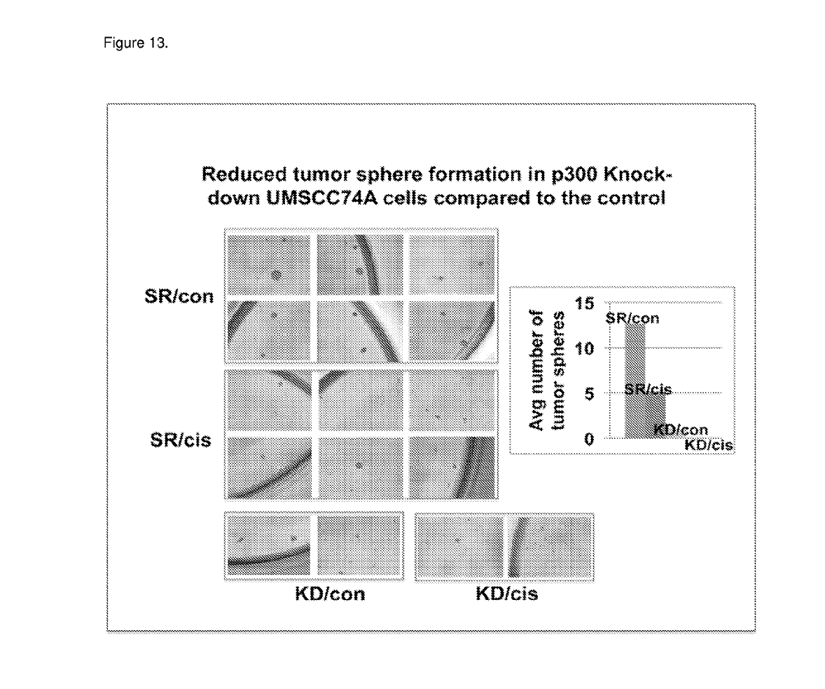

This example shows that Genetic knockdown of p300 in Head and Neck squamous cell carcinoma (HNSCC) leads to decreased Nanog expression and concomitant decrease in tumorsphere formation (this is in 3 different HNSCC cell lines) (see, e.g., FIG. 11. FIGS. 12 and 13 show impaired tumorsphere formation.

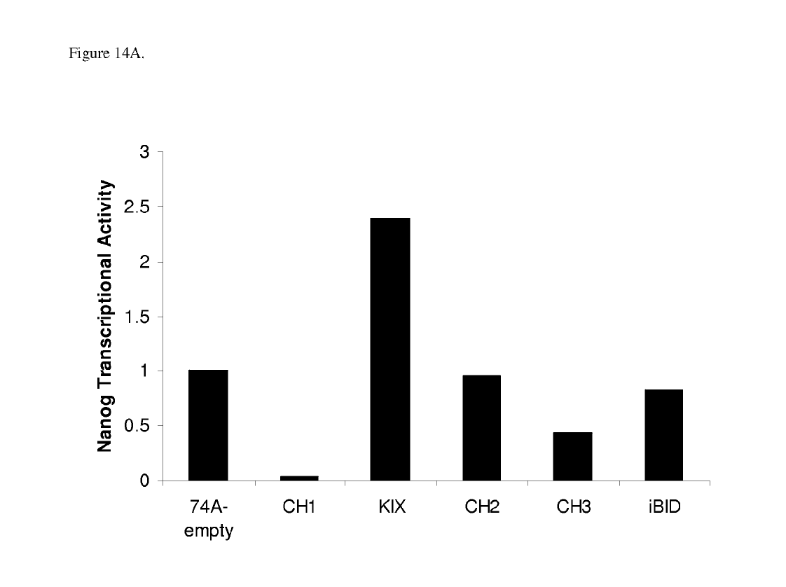

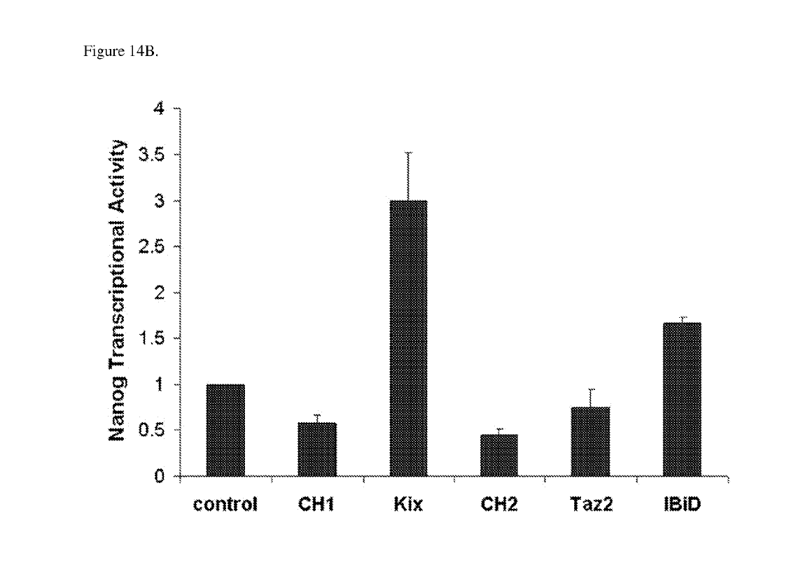

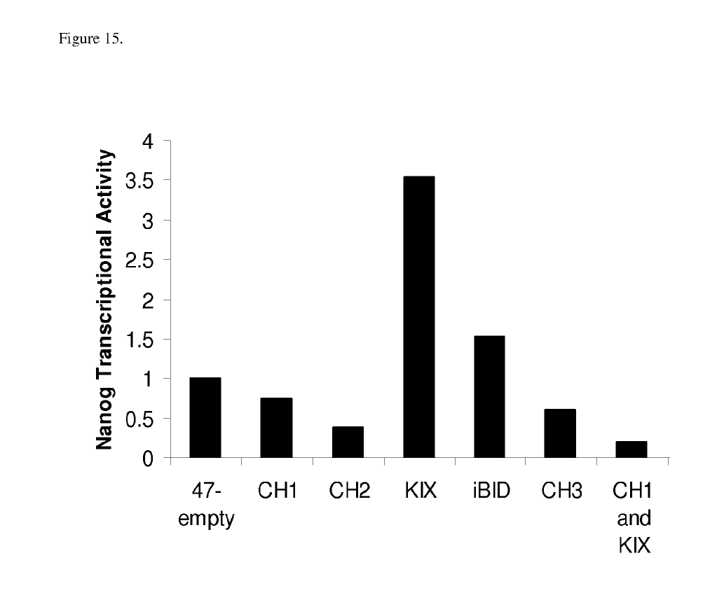

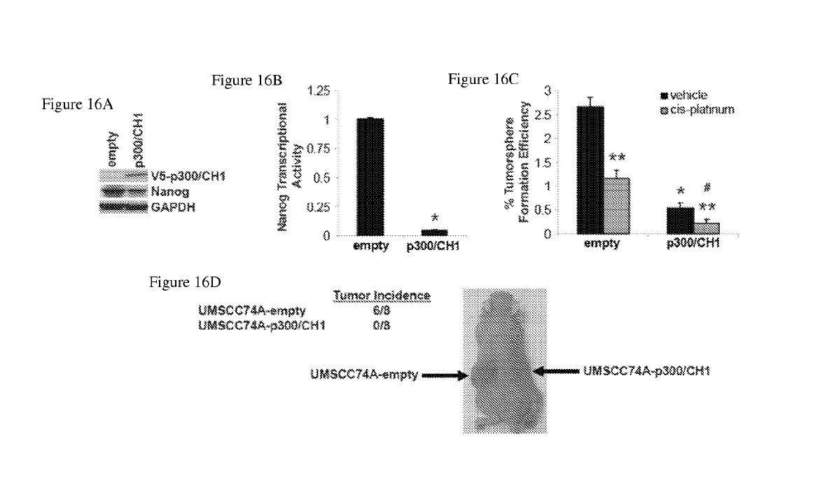

p300 is a multi-domain protein. A combination of deletion and overexpression studies conducted during development of embodiments for the present invention have shown that the KIX and CH1 domains alone and in combination have the most profound effects on Nanog expression in HNSCC cells. Consistent with these observations, deletion of the CH1 domain in NCCIT cells directly impairs Nanog expression but not Sox2 or Oct4. FIGS. 14A and B shows overexpression of the CH1 and KIX domains in HNSCC cells impacts Nanog transcriptional activity. FIG. 15 shows expression of CH1 and KIX domains in combination more effectively impacts Nanog transcriptional activity. FIG. 16 shows overexpression of the CH1 domain impacts Nanog expression, Nanog transcriptional activity, tumorsphere formation and tumor formation in mice. Moreover, molecules that interact with the KIX domain inhibit Nanog transcriptional activity with minimal impact on Oct4 and Sox2 in HNSCC cells. Molecules that interact with the KIX domain selectively impair the proliferation of HNSCC cancer stem cells. Molecules that interact with the KIX domain similar impair the proliferation of NCCIT cells.

### REFERENCES

1. Verdine, G. L.; Walensky, L. D. Clin Cancer Res 2007, 13, 7264-7270; herein incorporated by reference in its entirety.
2. Pandolfi, P. P. Oncogene 2001, 20, 3116-3127; herein incorporated by reference in its entirety.
3. Darnell, J. E., Jr. Nat Rev Cancer 2002, 2, 740-749; herein incorporated by reference in its entirety.
4. Koh, J. T.; Zheng, J. ACS Chem Biol 2007, 2, 599-601; herein incorporated by reference in its entirety.
5. Koehler, A. N. Curr Opin Chem Bio 2010, 14, 331-340; herein incorporated by reference in its entirety.
6. Lee, L. W.; Mapp, A. K. J Biol Chem 2010, 285, 11033-11038; herein incorporated by reference in its entirety.
7. Mapp, A. K.; Ansari, A. Z. ACS Chem Biol 2007, 2, 62-75; herein incorporated by reference in its entirety.
8. Vo, N.; Goodman, R. H. J Biol Chem 2001, 276, 13505-13508; herein incorporated by reference in its entirety.
9. Goodman, R. H.; Smolik, S. Genes Dev 2000, 14, 1553-1577; herein incorporated by reference in its entirety.
10. Kung, A. L.; Zabludoff, S. D.; France, D. S.; Freedman, S. J.; Tanner, E. A.; Vieira, A.; Cornell-Kennon, S.; Lee, J.; Wang, B.; Wang, J.; Memmert, K.; Naegeli, H. U.; Petersen, F.; Eck, M. J.; Bair, K. W.; Wood, A. W.; Livingston, D. M. Cancer Cell 2004, 6, 33-43; herein incorporated by reference in its entirety.
11. Liu, B.; Alluri, P. G.; Yu, P.; Kodadek, T. J Am Chem Soc 2005, 127, 8254-8255; herein incorporated by reference in its entirety.
12. Buhrlage, S. J.; Bates, C. A.; Rowe, S. P.; Minter, A. R.; Brennan, B. B.; Majmudar, C. Y.; Wemmer, D. E.; Al-Hashimi, H.; Mapp, A. K. ACS Chem Biol 2009, 4, 335-344; herein incorporated by reference in its entirety.
13. Cebrat, M.; Kim, C. M.; Thompson, P. R.; Daugherty, M.; Cole, P. A. Bioorg Med Chem 2003, 11, 3307-3313; herein incorporated by reference in its entirety.
14. Best, J. L.; Amezcua, C. A.; Mayr, B.; Flechner, L.; Murawsky, C. M.; Emerson, B.; Zor, T.; Gardner, K. H.; Montminy, M. Proc Natl Acad Sci USA 2004, 101, 17622-17627; herein incorporated by reference in its entirety.
15. Henchey, L. K.; Kushal, S.; Dubey, R.; Chapman, R. N.; Olenyuk, B. Z.; Arora, P. S. J Am Chem Soc 2010, 132, 941-943; herein incorporated by reference in its entirety.
16. Li, B. B. X.; Xiao, X. S. ChemBioChem 2009, 10, 2721-2724; herein incorporated by reference in its entirety.
17. Radhakrishnan, I.; Perez-Alvarado, G. C.; Parker, D.; Dyson, H. J.; Montminy, M. R.; Wright, P. E. Cell 1997, 91, 741-752; herein incorporated by reference in its entirety.
18. Radhakrishnan, I.; Perez-Alvarado, G. C.; Parker, D.; Dyson, H. J.; Montminy, M. R.; Wright, P. E. J Mol Biol 1999, 287, 859-865; herein incorporated by reference in its entirety.
19. Goto, N. K.; Zor, T.; Martinez-Yamout, M.; Dyson, H. J.; Wright, P. E. J Biol Chem 2002, 277, 43168-43174; herein incorporated by reference in its entirety.
20. Zor, T.; De Guzman, R. N.; Dyson, H. J.; Wright, P. E. J Mol Biol 2004, 337, 521-534; herein incorporated by reference in its entirety.
21. Campbell, K. M.; Lumb, K. J. Biochemistry 2002, 41, 13956-13946; herein incorporated by reference in its entirety.
22. Vendel, A. C.; Lumb, K. J. Biochemistry 2004, 43, 904-908; herein incorporated by reference in its entirety.
23. Vendel A. C.; McBryant, S. J.; Lumb K. J. Biochemistry, 2003, 12481-12487; herein incorporated by reference in its entirety.
24. Frangioni, J. V.; LaRiccia, L. M.; Cantley, L. C.; Montminy, M. R. Nat Biotechnol 2000, 18, 1080-1085; herein incorporated by reference in its entirety.
25. Rowe, S. P.; Mapp, A. K. Biopolymers 2008, 89, 578-581; herein incorporated by reference in its entirety.
26. Iyer, N. G.; Ozdag, H.; Caldas, C. Oncogene 2004, 23, 4225-4231; herein incorporated by reference in its entirety.
27. Kasper, L. H.; Fukuyama, T.; Biesen, M. A.; Boussouar, F.; Tong, C. L.; de Pauw, A.; Murray, P. J.; van Deursen, J. M. A.; Brindle, P. K. Mol Cell Biol 2006, 26, 789-809; herein incorporated by reference in its entirety.
28. Iyer, N. G.; Xian, J.; Chin, S. F.; Bannister, A. J.; Daigo, Y.; Aparicio, S.; Kouzarides, T.; Caldas, C. Oncogene 2007, 26, 21-29; herein incorporated by reference in its entirety.
29. Kimbrel, E. A.; Lemieux, M. E.; Xia, X. B.; Davis, T. N.; Rebel, V. I.; Kung, A. L. Blood 2009, 114, 4804-4812; herein incorporated by reference in its entirety.
30. Kinoshita, I.; Leaner, V.; Katabami, M.; Manzano, R. G.; Dent, P.; Sabichi, A.; Birrer, M. J. Oncogene 2003, 22, 2710-2722; herein incorporated by reference in its entirety.
31. Hanson, R. D.; Yu, B. D.; Hess, J.; Korsmeyer, S. J Blood 1997, 90, 184-194; herein incorporated by reference in its entirety.
32. Li, J. J.; Cao, Y.; Young, M. R.; Colburn, N. H. Mol Carcinogen 2000, 29, 159-169; herein incorporated by reference in its entirety.
33. Ayton, P. M.; Cleary, M. L. Gene Dev 2003, 17, 2298-2307; herein incorporated by reference in its entirety.
34. Leaner, V. D.; Kinoshita, I.; Birrer, M. J Oncogene 2003, 22, 5619-5629; herein incorporated by reference in its entirety.
35. Wisdom, R.; Johnson, R. S.; Moore, C. Embo J 1999, 18, 188-197; herein incorporated by reference in its entirety.
36. De Guzman, R. N.; Goto, N. K.; Dyson, H. J.; Wright, P. E. J Mol Biol 2006, 355, 1005-1013; herein incorporated by reference in its entirety.
37. Minter, A. R.; Brennan, B. B.; Mapp, A. K. J Am Chem Soc 2004, 126, 10504-10505; herein incorporated by reference in its entirety.
38. Buhrlage, S. J.; Brennan, B. B.; Minter, A. R.; Mapp, A. K. J Am Chem Soc 2005, 127, 12456, 12457; herein incorporated by reference in its entirety.
39. Rowe, S. P.; Casey, R. J.; Brennan, B. B.; Buhrlage, S. J.; Mapp, A. K. J Am Chem Soc 2007, 129, 10654-10655; herein incorporated by reference in its entirety.
40. Naidu, S. R.; Love, I. M.; Imbalzano, A. N.; Grossman, S. R.; Androphy, E. J. Oncogene 2009, 28, 2492-2501; herein incorporated by reference in its entirety.
41. Bannister, A. J.; Kouzarides, T. Embo J 1995, 14, 4758-4762; herein incorporated by reference in its entirety.
42. Green, M.; Panesar, N. K.; Loewenstein, P. M. Oncogene 2008, 27, 4446-4455; herein incorporated by reference in its entirety.
43. Peter Pelka, J. N. G. A., Joseph, T.; Andrew, S. T.; Roger, J. A. G.; Joe, S. M. Nuc Acids Res 2009, 37, 12, 1095-1106.44. Ernst, P.; Wang, J.; Huang, M.; Goodman, R. H.; Korsmeyer, S. J. Mol Cell Biol 2001, 21, 2249-2258; herein incorporated by reference in its entirety.
45. Lee, L. W.; Taylor, C. E. C.; Desaulniers, J. P.; Zhang, M. C.; Hojfeldt, J. W.; Pan, Q.; Mapp, A. K. Bioorg Med Chem Lett 2009, 19, 6233-6236; herein incorporated by reference in its entirety.
46. Chang, C. H.; Scott, G. K.; Kuo, W. L.; Xiong, X. H.; Suzdaltseva, Y.; Park, J. W.; Sayre, P.; Erny, K.; Collins, C.; Gray, J. W.; Benz, C. C. Oncogene 1997, 14, 1617-1622; herein incorporated by reference in its entirety.
47. Kim, J. K.; Diehl, J. A. J Cell Phys 2009, 220, 292-296; herein incorporated by reference in its entirety.
48. Fu, M. F.; Wang, C. G.; Li, Z. P.; Sakamaki, T.; Pestell, R. G. Endocrinology 2004, 145, 5439-5447; herein incorporated by reference in its entirety.
49. Arnold, A.; Papanikolaou, A. J Clin Oncol 2005, 23, 4215-4224; herein incorporated by reference in its entirety.
50. Tashiro, E.; Tsuchiya, A.; Imoto, M. Cancer Sci 2007, 98, 629-635; herein incorporated by reference in its entirety.
51. Stacey, D. W. Curr Opin in Cell Biol 2003, 15, 158-163; herein incorporated by reference in its entirety.
52. Albanese, C.; D'Amico, M.; Reutens, A. T.; Fu, M.; Watanabe, G.; Lee, R. J.; Kitsis, R. N.; Henglein, B.; Avantaggiati, M.; Somasundaram, K.; Thimmapaya, B.; Pestell, R. G. J. Biol Chem 1999, 274, 34186-34195; herein incorporated by reference in its entirety.
53. Bakiri, L.; Lallemand, D.; Bossy-Wetzel, E.; Yaniv, M. Embo J 2000, 19, 2056-2068; herein incorporated by reference in its entirety.
54. Amanatullah, D. F.; Zafonte, B. T.; Albanese, C.; Fu, M. F.; Messiers, C.; Hassell, J.; Pestell, R. G. Methods Enzymol 2001, 333, 116-127; herein incorporated by reference in its entirety.
55. Casey, R. J.; Desaulniers, J. P.; Hojfeldt, J. W.; Mapp, A. K. Bioorgan Med Chem 2009, 17, 1034-1043; herein incorporated by reference in its entirety.
56. Cochran, A. G. Chem Biol 2000, 7, R85-R94; herein incorporated by reference in its entirety.
57. Berg, T. Angewandte Chemie-Int Ed 2003, 42, 2462-2481; herein incorporated by reference in its entirety.
58. Arkin, M. R.; Wells, J. A. Nat Rev Drug Disc 2004, 3, 301-317; herein incorporated by reference in its entirety.
59. Fletcher, S.; Hamilton, A. D. Curr Opin Chem Biol 2005, 9, 632-638; herein incorporated by reference in its entirety.
60. Bannister, A. J.; Kouzarides, T. Nature 1996, 384, 641-643; herein incorporated by reference in its entirety.
61. Ogryzko, V. V.; Schiltz, R. L.; Russanova, V.; Howard, B. H.; Nakatani, Y. Cell 1996, 87, 953-959; herein incorporated by reference in its entirety.
62. Marian A. Martinez-Balbas, A. J. B.; Klaus Martin, P. H.- S.; Michael, M.; Tony, K. EMBO J 1998, 17, 6, 2886-2893; herein incorporated by reference in its entirety.
63. Chrivia, J. C.; Kwok, R. P. S.; Lamb, N.; Hagiwara, M.; Montminy, M. R.; Goodman, R. H. Nature 1993, 365, 855-859; herein incorporated by reference in its entirety.
64. Samuelson, A. V.; Lowe, S. W. Proc Natl Acad Sci USA 1997, 94, 12094-12099.
65. Ptashne, M.; Gann, A. Nature 1997, 386, 569-577; herein incorporated by reference in its entirety.
66. Ansari, A. Z.; Mapp, A. K.; Nguyen, D. H.; Dervan, P. B.; Ptashne, M. Chem Biol 2001, 8, 583-592; herein incorporated by reference in its entirety.
67. Still, W. C.; Kahn, M.; Mitra, A. J Org Chem 1978, 43, 2923-2925; herein incorporated by reference in its entirety.
68. Singh S K, Clarke I D, Terasaki M, Bonn V E, Hawkins C, Squire J, Dirks P B (September 2003). “Identification of a cancer stem cell in human brain tumors”. Cancer research 63 (18): 5821-8.
69. Al-Hajj M, Wicha M S, Benito-Hernandez A, Morrison S J, Clarke M F (April 2003). “Prospective identification of tumorigenic breast cancer cells”. Proceedings of the National Academy of Sciences of the United States of America 100 (7): 3983-8.
70. O'Brien C A, Pollett A, Gallinger S, Dick J E (January 2007). “A human colon cancer cell capable of initiating tumour growth in immunodeficient mice”. Nature 445 (7123): 106-10.
71. Zhang S, Balch C, Chan M W, Lai H C, Matei D, Schilder J M, Yan P S, Huang T H, Nephew K P (June 2008). “Identification and characterization of ovarian cancer-initiating cells from primary human tumors”. Cancer research 68 (11): 4311-20.
72. Li C, Heidt D G, Dalerba P, Burant C F, Zhang L, Adsay V, Wicha M, Clarke M F, Simeone D M (February 2007). “Identification of pancreatic cancer stem cells”. Cancer research 67 (3): 1030-7.
73. Maitland N J, Collins A T (June 2008). “Prostate cancer stem cells: a new target for therapy”. J. Clin. Oncol. 26 (17): 2862-70.
74. Lang Sh, Frame F, Collins A (January 2009). “Prostate cancer stem cells”. J. Pathol. 217 (2): 299-306.
75. Schatton T, Murphy G F, Frank, N Y, Yamaura K, Waaga-Gasser A M, Gasser M, Zhan Q, Jordan S, Duncan L M, Weishaupt C, Fuhlbrigge R C, Kupper T S, Sayegh M H, Frank M H (January 2008). “Identification of cells initiating human melanomas”. Nature 451 (7176): 345-9.
76. Boiko A D, Razorenova O V, van de Rijn M, Swetter S M, Johnson D L, Ly D P, Butler P D, Yang G P, Joshua B, Kaplan M J, Longaker M T, Weissman I L (July 2010). “Human melanoma-initiating cells express neural crest nerve growth factor receptor CD271”. Nature 466 (7302): 133-7.
77. Schmidt P, Kopecky C, Hombach A, Zigrino P, Mauch C, Abken H. (February 2011). “Eradication of melanomas by targeted elimination of a minor subset of tumor cells.”. PNAS 108 (6): 2474-9.
78. Civenni G, Walter A, Kobert N, Mihic-Probst D, Zipser M, Belloni B, Seifert B, Moch H, Dummer R, van den Broek M, Sommer L. (March 2011). “Human CD271-Positive Melanoma Stem Cells Associated with Metastasis Establish Tumor Heterogeneity and Long-Term Growth”. Cancer Res. 71 (8): 3098-109.
79. Matsui W, Huff C A, Wang Q, et al. (March 2004). “Characterization of clonogenic multiple myeloma cells”. Blood 103 (6): 2332-6.
80. Matsui W, Wang Q, Barber J P, et al. (January 2008). “Clonogenic multiple myeloma progenitors, stem cell properties, and drug resistance”. Cancer Res. 68 (1): 190-7.
81. Park I K, Qian D, Kiel M, Becker M W, Pihalja M, Weissman I L, Morrison S J, Clarke M F (May 2003). “Bmi-1 is required for maintenance of adult self-renewing haematopoietic stem cells”. Nature 423 (6937): 302-5.
82. Hemmati H D, Nakano I, Lazareff J A, Masterman-Smith M, Geschwind D H, Bronner-Fraser M, Kornblum H I (December 2003). “Cancerous stem cells can arise from pediatric brain tumors”. Proceedings of the National Academy of Sciences of the United States of America 100 (25): 15178-83.
83. Diévart A, Beaulieu N, Jolicoeur P (October 1999). “Involvement of Notch1 in the development of mouse mammary tumors”. Oncogene 18 (44): 5973-81.
84. Gupta, et al., 2009, Identification of Selective Inhibitors of Cancer Stem Cells by High-Throughput Screening, Cell 138, 645-659.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1.-48. (canceled)

49. A method for regulating CBP-mediated transcription of a gene of interest, comprising:
a) providing
i) host cells expressing: CREB-binding protein (CBP) comprising a KIX domain, a coactivator protein known to bind the KIX domain within CBP, and a gene of interest, wherein binding of said coactivator protein within said KIX domain is required for said CBP-mediated transcription of said gene of interest, wherein said host cells are ex vivo host cells and/or cancer cells, wherein said coactivator protein is selected from the group consisting of MLL, Jun, Tat, and Tax; and
ii) small molecules capable of binding within said KIX domain;
b) delivering to said host cells an effective amount of said small molecules such that expression of said gene of interest is modified.

50. The method of claim 49, wherein said small molecules are isoxazolidine compounds.

51. The method of claim 50, wherein said isoxazolidine compounds are represented by the following formula: 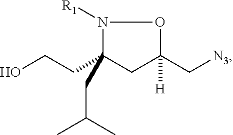
including salts, esters and prodrugs thereof, wherein R1 is a functional group facilitating binding within said KIX domain.

52. The method of claim 51, wherein R1 is configured to mimic at least a portion of amino acid residues selected from the group consisting of:
amino acid residues 2840-2858 (ILPSDIMDFLVKNTP) (SEQ ID NO:1) within MLL,
amino acid residues 47-66 (VLLKLASPELERLIIQSSN) (SEQ ID NO:2) within Jun, amino acid residues 1-24 (MEPVDPRLEPWKHPGSQPKT) (SEQ ID NO:3) within Tat, and
amino acid residues 76-95 (PSFPTQRTSKTLKVLPPIT) (SEQ ID NO: 4) within Tax.

53. The method of claim 51, wherein R1 is selected from the group consisting of

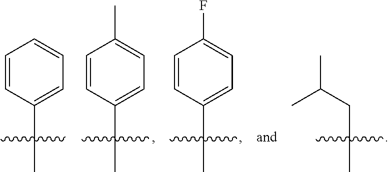

54. The method of claim 49, wherein said small molecule is selected from the group consisting of

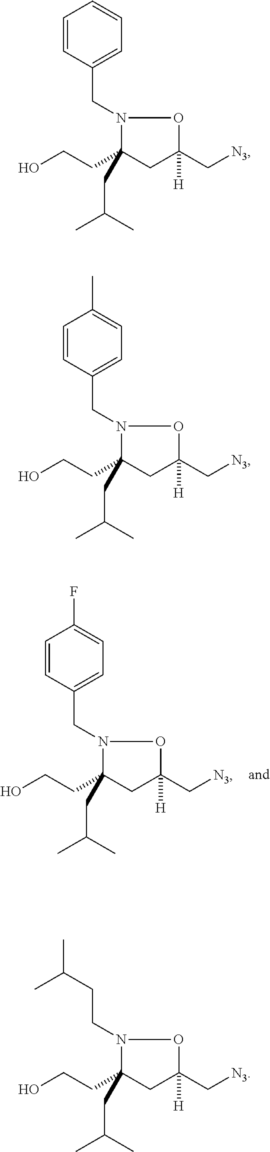 [SMILES: CC(C)C[C@]1(CCO)C[C@H](CN=[N+]=[N-])ON1Cc1ccccc1 ; Cc1ccc(CN2O[C@@H](CN=[N+]=[N-])C[C@@]2(CCO)CC(C)C)cc1 ; CC(C)C[C@]1(CCO)C[C@H](CN=[N+]=[N-])ON1Cc1ccc(F)cc1 ; CC(C)CCN1O[C@@H](CN=[N+]=[N-])C[C@@]1(CCO)CC(C)C]

55. A method for regulating p300-mediated transcription of a gene of interest, comprising:
a) providing
i) host cells expressing: p300 comprising a KIX domain and a CH1 domain, a coactivator protein known to bind the KIX and/or CH1 domains within p300, and a gene of interest, wherein binding of said coactivator protein within said KIX and/or CH1 domains is required for said CBP-mediated transcription of said gene of interest, wherein said coactivator protein is Nanog, wherein said host cells are ex vivo host cells and/or cancer cells, wherein said cancer cells are selected from the group consisting of HNSCC cells and NCCIT cells; and
ii) small molecules capable of binding within said KIX and/or CH1 domains;
b) delivering to said host cells an effective amount of said small molecules such that expression of said gene of interest is modified.

56. The method of claim 55, wherein said small molecules are isoxazolidine compounds.

57. The method of claim 56, wherein said isoxazolidine compounds are represented by the following formula: 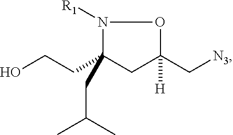
including salts, esters and prodrugs thereof, wherein R1 is a functional group facilitating binding within said KIX and/or CH1 domains.

58. The method of claim 55, wherein said small molecule is

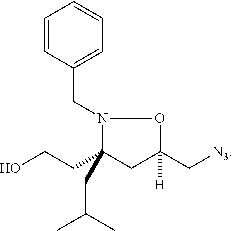 [SMILES: CC(C)C[C@]1(CCO)C[C@H](CN=[N+]=[N-])ON1Cc1ccccc1]

59. A method for treating a human subject having a disorder comprising administering to said subject a pharmaceutical composition,
wherein said disorder and pharmaceutical composition are selected from the group consisting of
a disorder having aberrant CBP related transcription and a pharmaceutical composition comprising a CBP-mediated transcription inhibitor, and
a disorder having aberrant p300 related transcription and a pharmaceutical composition comprising a p300-mediated transcription inhibitor.

60. The method of claim 59,
wherein said CBP related transcription is selected from the group consisting of MLL*CBP related transcription and Jun*CBP related transcription,
wherein said p300 related transcription is Nanog*p300 related transcription.

61. The method of claim 59,
wherein said disorder having aberrant CBP related transcription is leukemia and/or a solid tumor based cancer,
wherein said disorder having aberrant p300 related transcription is head and neck squamous cell carcinoma (HNSCC).

62. The method of claim 59,
wherein said CBP-mediated transcription inhibitor is an isoxazolidine compound,
wherein said p300-mediated transcription inhibitor is an isoxazolidine compound.

63. The method of claim 62, wherein said isoxazolidine compound is represented by the following formula: 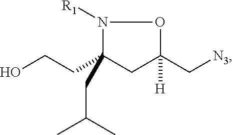
including salts, esters and prodrugs thereof,
wherein R1 is a functional group facilitating binding of said isoxazolidine compound with a CREB-binding protein (CBP) comprising a KIX domain, wherein said binding occurs within said KIX domain, and/or
wherein R1 is a functional group facilitating binding of said isoxazolidine compound with a p300 protein comprising a CH1 domain, wherein said binding occurs within said CH1 domain.

64. The method of claim 62,
wherein said isoxazolidine compound is configured to mimic a coactivator protein selected from the group consisting of Nanog, MLL, Jun, Tat, and Tax.

65. The method of claim 64,
wherein said isoxazolidine compound is configured to mimic at least a portion of amino acid residues selected from the group consisting of:
amino acid residues 2840-2858 (ILPSDIMDFLVKNTP) (SEQ ID NO:1) within MLL,
amino acid residues 47-66 (VLLKLASPELERLIIQSSN) (SEQ ID NO:2) within Jun, amino acid residues 1-24 (MEPVDPRLEPWKHPGSQPKT) (SEQ ID NO:3) within Tat, and
amino acid residues 76-95 (PSFPTQRTSKTLKVLPPIT) (SEQ ID NO: 4) within Tax.

66. The method of claim 63, wherein said small molecule is selected from the group consisting of

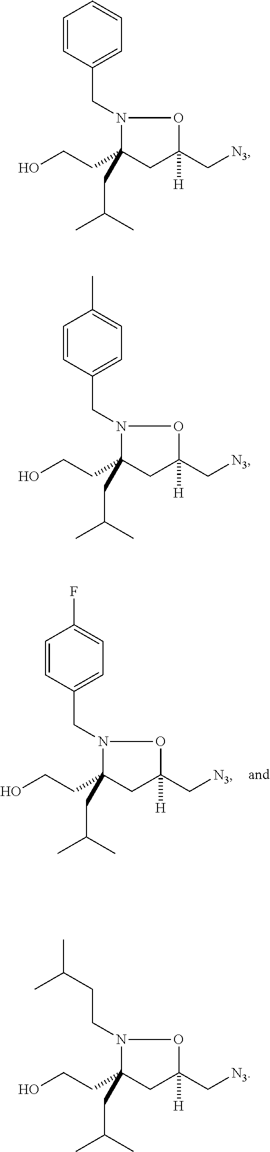 [SMILES: CC(C)C[C@]1(CCO)C[C@H](CN=[N+]=[N-])ON1Cc1ccccc1 ; Cc1ccc(CN2O[C@@H](CN=[N+]=[N-])C[C@@]2(CCO)CC(C)C)cc1 ; CC(C)C[C@]1(CCO)C[C@H](CN=[N+]=[N-])ON1Cc1ccc(F)cc1 ; CC(C)CCN1O[C@@H](CN=[N+]=[N-])C[C@@]1(CCO)CC(C)C]

67. The method of claim 62, further comprising co-administering to the subject effective amounts of one or more anti-cancer therapeutic agents.

68. The method of claim 59, wherein said disorder comprises proliferating cancer-initiating cells.
